SEQUENCE LISTING (20 sequences)

SEQ ID NO: 1 - DNA, 1627 bp, Homo sapiens
  CDS (79)..(1185): transmembrane protein with EGF-like and two follistatin-like domains 2 (TMEFF2). Uchida, et al. Biochem. Biophys. Res. Commun. 602 (1999), 266593; Horie, et al. Genomics 67 (2000),146-152
cagtagcccgctgcccggcccccgcgatcctgtgttcctcggaagccgtttgctgctgcagagttgcacgaactagtcatggtgctgtgggagtccccgcggcagtgcagcagctggacactttgcgagggcttttgctggctgctgctgctgcccgtcatgctactcatcgtagcccgcccggtgaagctcgctgctttccctacctccttaagtgactgccaaacgcccaccggctggaattgctctggttatgatgacagagaaaatgatctcttcctctgtgacaccaacacctgtaaatttgatggggaatgtttaagaattggagacactgtgacttgcgtctgtcagttcaagtgcaacaatgactatgtgcctgtgtgtggctccaatggggagagctaccagaatgagtgttacctgcgacaggctgcatgcaaacagcagagtgagatacttgtggtgtcagaaggatcatgtgccacagatgcaggatcaggatctggagatggagtccatgaaggctctggagaaactagtcaaaaggagacatccacctgtgatatttgccagtttggtgcagaatgtgacgaagatgccgaggatgtctggtgtgtgtgtaatattgactgttctcaaaccaacttcaatcccctctgcgcttctgatgggaaatcttatgataatgcatgccaaatcaaagaagcatcgtgtcagaaacaggagaaaattgaagtcatgtctttgggtcgatgtcaagataacacaactacaactactaagtctgaagatgggcattatgcaagaacagattatgcagagaatgctaacaaattagaagaaagtgccagagaacaccacataccttgtccggaacattacaatggcttctgcatgcatgggaagtgtgagcattctatcaatatgcaggagccatcttgcaggtgtgatgctggttatactggacaacactgtgaaaaaaaggactacagtgttctatacgttgttcccggtcctgtacgatttcagtatgtcttaatcgcagctgtgattggaacaattcagattgctgtcatctgtgtggtggtcctctgcatcacaaggaaatgccccagaagcaacagaattcacagacagaagcaaaatacagggcactgtgggtataatactaagttgagatgatatcatttacgggggaaggcgctttgtgaagtaggccttatttctcttgtcctttcgtacagggaggaatttgaagtagatagaaaccgacctggattactccggtctgaactcagatcacgtaggactttaatcgttgaacaaacgaacctttaatagcggctgcaccatcgggatgtcctgatccaacatcgaggtcgtaaaccctattgttgatatggactctagaataggattgcgctgttatccctagggtaacttgttccgttggtcaagttattggatcaattgagtatagtagttcgctttgactggtgaagtcttagcatgtactgctcggaggttgggttctgctccgaggtcgccccaaccgaaatttttaatgcaggtttggtagtttaggacctgtgggtttgttaggtactgtttgcattaataaatt

SEQ ID NO: 2 - protein, 368 aa, Homo sapiens [sequence omitted]

SEQ ID NO: 3 - protein, 5 aa, Artificial Sequence
  synthetic peptide
DTYMH

SEQ ID NO: 4 - protein, 17 aa, Artificial Sequence
  synthetic peptide
RIDPANGNTKYDPNFQG

SEQ ID NO: 5 - protein, 12 aa, Artificial Sequence
  synthetic peptide
WGYYGTSGYFDV

SEQ ID NO: 6 - protein, 17 aa, Artificial Sequence
  synthetic peptide
KSSQSLLSSRTRKNYLA

SEQ ID NO: 7 - protein, 7 aa, Artificial Sequence
  synthetic peptide
WASTRES

SEQ ID NO: 8 - protein, 8 aa, Artificial Sequence
  synthetic peptide
KQSYNLWT

SEQ ID NO: 9 - DNA, 363 bp, Mus musculus
  CDS (1)..(363): PQ01 Heavy chain variable region (VH) sequence
gaggtgcagctgcaggagtctggggccgagcttgtgaagccaggggcctcagtcaagttgtcctgcacagcttctggcttcaacattaaagacacctatatgcactgggtgaagcagaggcctgaacagggcctggagtggattggaaggattgatcctgcgaatggtaatactaaatatgacccgaacttccagggcaaggccactattacatcagacacatcctccaacacagcctacctgcagctcagcagcctgacatctgaggacactgccgtctattactgtgctagatgggggtactacggtactagcgggtacttcgatgtctggggccaagggaccacggtcaccgtttcctca

SEQ ID NO: 10 - protein, 121 aa, Mus musculus
EVQLQESGAELVKPGASVKLSCTASGFNIKDTYMHWVKQRPEQGLEWIGRIDPANGNTKYDPNFQGKATITSDTSSNTAYLQLSSLTSEDTAVYYCARWGYYGTSGYFDVWGQGTTVTVSS

SEQ ID NO: 11 - DNA, 339 bp, Mus musculus
  CDS (1)..(339): PQ01 VL variable light chain (VL) sequence
gacattgagctcacccagtctccagcctccctggctgtgtcagcaggagagaaggtcactatgagctgcaaatccagtcagagtctgctcagcagtagaacccgaaagaactacttggcttggtaccagcagaaaccagggcagtctcctaaactgctgatctactgggcatccactcgggaatctggggtccctgatcgcttcacaggcagtggatctgggacagatttcactctcaccatcagcagtgtgcaggctgaagacctggcagtttattactgcaagcaatcttataatctgtggacgttcggagcagggaccaagctcgagctgaaacgg

SEQ ID NO: 12 - protein, 113 aa, Mus musculus
DIELTQSPASLAVSAGEKVTMSCKSSQSLLSSRTRKNYLAWYQQKPGQSPKLLIYWASTRESGVPDRFTGSGSGTDFTLTISSVQAEDLAVYYCKQSYNLWTFGAGTKLELKR

SEQ ID NO: 13 - protein, 7 aa, Artificial Sequence
  synthetic peptide
EDGHYAR

SEQ ID NO: 14 - protein, 15 aa, Artificial Sequence
  synthetic peptide
NTTTTTKSEDGHYAR

SEQ ID NO: 15 - protein, 15 aa, Artificial Sequence
  synthetic peptide
TTKSEDGHYARTDYA

SEQ ID NO: 16 - protein, 15 aa, Artificial Sequence
  synthetic peptide
EDGHYARTDYAENAN

SEQ ID NO: 17 - protein, 23 aa, Artificial Sequence
  synthetic peptide
NTTTTTKSEDGHYARTDYAENAN

SEQ ID NO: 18 - protein, 97 aa, Artificial Sequence
  synthetic peptide
QFGAECDEDAEDVWCVCNIDCSQTNFNPLCASDGKSYDNACQIKEASCQKQEKIEVMSLGRCQDNTTTTTKSEDGHYARTDYAENANKLEESAREHH

SEQ ID NO: 19 - protein, 11 aa, Artificial Sequence
  synthetic peptide
ACTTPHAWLCG

SEQ ID NO: 20 - protein, 92 aa, Artificial Sequence
  synthetic peptide
SYDNACQIKEASCQKQEKIEVMSLGRCQDNTTTTTKSEDGHYARTDYAENANKLEESAREHHIPCPEHYNGFCMHGKCEHSINMQEPSCRCD

(19) United States
(12) Patent Application Publication
(10) Pub. No.: US 2015/0030602 A1
(43) Pub. Date: Jan. 29, 2015

(54) ANTIBODIES FOR THE TREATMENT AND DIAGNOSIS OF AFFECTIVE AND ANXIETY DISORDERS

(71) Applicants: Ingeborg Sillaber, Munich (DE); Marcelo Paez-Pereda, Munich (DE)

(72) Inventors: Ingeborg Sillaber, Munich (DE); Marcelo Paez-Pereda, Munich (DE)

(73) Assignee: PHENOQUEST AG, Martinsried (DE)

(21) Appl. No.: 14/364,590

(22) PCT Filed: Jan. 2, 2013
(86) PCT No.: PCT/EP2013/050003

§ 371 Date: Jun. 11, 2014

Related U.S. Application Data
(60) Provisional application No. 61/741,790, filed on Dec. 23, 2011.

(30) Foreign Application Priority Data
Jul. 12, 2012 (EP) 12176213.2

(51) Int. Cl. C07K 16/28; G01N 33/68

(52) U.S. Cl. CPC C07K 16/28; G01N 33/68; G01N 2333/705; C07K 2317/565; C07K 2317/55; C07K 2317/622; C07K 2317/76; A61K 2039/505
USPC 424/139.1; 530/387.9; 530/387.3; 530/391.3; 530/391.7; 536/23.53; 435/320.1; 435/252.33; 435/252.31; 435/252.3; 435/254.2; 435/419; 435/331; 435/6.19; 435/7.92; 435/188

(57) ABSTRACT

A novel monoclonal antibody and like antigen-binding molecules against transmembrane protein with EGF-like and two follistatin-like domains 2 (TMEFE2) are provided with unique immunological and biological properties useful in the therapy of affective disorders such as depression and bipolar disorders as well as anxiety disorders. In addition, pharmaceutical compositions and kits comprising such antibody and derivatives thereof are described.

### FIELD OF THE INVENTION

The present invention generally relates to novel anti-TMEFF2 (Transmembrane protein with EGF-like and two follistatin-like domains 2)-specific binding molecules, particularly monoclonal antibodies as well as fragments, derivatives and variants thereof that recognize TMEFF2. In addition, the present invention relates to pharmaceutical and diagnostic compositions comprising such binding molecules, antibodies and mimics thereof valuable both as a diagnostic tool to identify and for treating, respectively, disorders related to affective disorders, such as depression, and anxiety.

### BACKGROUND OF THE INVENTION

Up to 10% of persons visiting a physician are afflicted with an affective disorder (also known as behavioral disorder, mood disorder) or anxiety disorders. Nonetheless, most cases remain undiagnosed or inadequately treated. Affective disorders include among others, depression and bipolar disorder. Affective and anxiety disorders are well described in the literature; see, for example, International Statistical Classification of Diseases and Related Health Problems 10th Revision (ICD-10, Version 2010, WHO, F20-F48) or the Diagnostic and Statistical Manual of Mental Disorders-4th Edition Text Revision (DSM-IV-TR), American Psychiatric Press, 2000. Present treatment of depression consists of psychotherapy, antidepressant drugs or a combination of both. Most antidepressant drugs target the transport of the neurotransmitters serotonin and/or norepinephrine, or the activity of the enzyme monoamine oxidase. However, all existing antidepressant drugs possess shortcomings such as long latency until response, high degree of non-responders, and undesirable side effects (Holsboer, Biol. Psychol. 57 (2001), 47-65).

Accordingly, there is a need for new anti-depressive drugs with different mechanisms of action and improved pharmacological profile (Baldwin, Hum. Psychopharmacol. Clin. Exp. 16 (2001):S93-S99; Greden, J. Clin. Psychiatry 63 Suppl. 2 (2002):3-7).

### SUMMARY OF THE INVENTION

The present invention generally relates to compounds having anti-depressive and/or anxiolytic activity leaving normal behavior of the subject to be treated unaffected in kind. In particular, a monoclonal antibody and like binding molecules are provided capable of specifically binding transmembrane protein with EGF-like and two follistatin-like domains 2 (TMEFF2), characterized in that the antibody is capable of increasing Activin induced Smad-regulated signaling pathway activity in TMEFF2 over-expressing CHO cells, displaying anxiolytic properties in the novelty-induced hypophagia (NIH) paradigm test and displaying anti-depressive properties in the forced swim test (FST) in an appropriate animal model while preferably the locomotor activity of the animals remains substantially unchanged. Furthermore, the present invention relates to compositions comprising said compounds and to immunotherapeutic and immunodiagnostic methods using the same.

In a particularly preferred embodiment, the antibody and antigen-binding molecule, respectively, demonstrates one or more of the immunological binding characteristics and/or biological activities of the monoclonal antibody characterized by the variable regions VH and/or VL as set forth in FIG. 1, infra, and further described in the Examples.

Alternatively, the antibody is a human, humanized, xenogeneic, or a chimeric human-murine antibody, the latter being particularly useful for diagnostic methods and studies in animals. Compositions including the antibody or active fragments thereof, or agonists and cognate molecules, or alternatively, antagonists of the same, and methods of use of such compositions in the prevention, diagnosis or treatment of a disorder using these compositions are also included, wherein an effective amount of the composition is administered to a patient in need of such treatment.

The antibody and antigen-binding molecule such as antigen-binding fragment of the subject antibody can be a single chain Fv fragment, an F(ab′) fragment, an F(ab) fragment, and an F(ab′)₂ fragment, or any other antigen-binding fragment. In a specific embodiment, infra, the monoclonal antibody or fragment thereof is a murine IgG isotype antibody.

Naturally, the present invention extends to the hybridoma that produces the monoclonal antibody as well as to genetically engineered cell lines expressing the recombinant antibody having the distinct and unique characteristics as defined below.

The present invention also relates to polynucleotides encoding at least a variable region of an immunoglobulin chain of the antibody of the invention. Preferably, said variable region comprises at least one complementarity determining region (CDR) of the V_{H} and/or V_{L} of the variable region as set forth in FIG. 1, infra.

Accordingly, the present invention also encompasses vectors comprising said polynucleotides and host cells transformed therewith as well as their use for the production of an antibody and equivalent binding molecules which are specific for TMEFF2, in particular specific for the epitope region identified for the subject antibody in the Examples, i.e. the region between the Kazal-like 2 and EGF-like domain.

The antibody, immunoglobulin chain(s), binding fragments thereof, and ligands other than TMEFF2-binding molecules having equivalent binding to the antibody illustrated in the Examples can be used in pharmaceutical and diagnostic compositions for immunotherapy and diagnosis, respectively. The use of the foregoing compositions in the preparation of a medicament is however preferred.

Hence, it is a particular object of the present invention to provide methods for treating or preventing an affective disorder or for diagnosing or screening a subject for the presence or for determining a subject's risk for developing an affective disorder such as major depression, dysthymia, atypical depression, premenstrual dysphoric disorder, seasonal affective disorder, and bipolar disorder. The methods comprise administering an effective amount of the compound of the present invention, in particular the subject antibody or a derivative thereof to a subject in need thereof. In addition, the compounds and compositions described herein can be used to identify pre-symptomatic disease, monitor disease progression and therapeutic efficacy of putative psychopharmacological drugs and other agents for the treatment of affective and/or anxiety disorders. In a preferred embodiment of the present invention the disorder is depression and generalized anxiety.

Due to the provision of the subject antibodies having unique properties and in particular by disclosing the variable region and complementarity determining region (CDRs) of the subject antibody as well as the epitope recognized by the antibody the present invention also provides the necessary structural information for and thus extends to anti-idiotypic antibodies and (synthetic) peptides or peptide-based compounds comprising an epitope specifically recognized by an antibody of the present invention. Anti-idiotypic antibodies and equivalent binding molecules as well as epitopes of the present invention are particularly useful for diagnostic purposes, for example in the detection and isolation of protective anti-TMEFF2 autoantibodies in humans.

Further embodiments of the present invention will be apparent from the description that follows.

### BRIEF DESCRIPTION OF THE DRAWINGS

FIG. 1: Amino acid sequences of the variable light chain and heavy chain of antibody PQ01 with complementarity determining regions (CDRs) as determined according to Kabat underlined as well as the corresponding DNA sequences. VL, variable light chain; VH, variable heavy chain; CDRL, CDR of the variable light chain; CDRH, CDR of the variable heavy chain.

FIG. 2: Western Blot analysis of anti-TMEFF2 antibody; see Example 1.

FIG. 3: Exemplary ELISA assay result for anti-TMEFF2 antibodies producing hybridoma subclones generated in Example 1. The TMEFF2 protein fragment (97aa) used for immunizing the mice was used as the antigen to be screened against.

FIG. 4: Binding of anti-TMEFF2 antibodies as obtained in the screening of Example 1 at different concentrations (0-500 ng/ml) to the 97aa TMEFF2 peptide used for antibody production (immunization), determined by ELISA. Antibodies: #001 (PQ01), #13n, #14v, #31s.

FIG. 5: Binding of TMEFF2 antibodies (500 ng/ml) to the full-length TMEFF2 protein (Abnova, #H00023671-P01), determined by ELISA. PQ01: own antibody; ab77038: TMEFF2 antibody from abcam (#ab77038); 1D12: TMEFF2 antibody from Abnova (#H00023671-M08); J4B6: TMEFF2 antibody from Abnova (#MAB2055); see Example 2.

FIG. 6: Signals yielded after incubation with target antibody PQ01 in the epitope mapping experiment described in Example 3.

FIG. 7: Systemic injection of TMEFF2 antibody PQ01 displayed antidepressant-like effects in female DBA/2JIco mice in the forced swim test (FST) paradigm. PQ01 reduces passive and increases active escape-oriented behaviour in the FST. The times the mice spent with struggling, swimming and floating during the 5 minutes test period is shown. *P<0.05, Mann-Whitney U test. N=10 per group.

FIG. 8: Systemic injection of TMEFF2 antibody PQ01 left general locomotor activity unchanged in female DBA/2JIco mice. Locomotion (m) was assessed in the open field test (OF) and data per 5 minutes interval are shown. N=10 per group.

FIG. 9: Systemic injection of the anti-TMEFF2 antibody PQ01 displayed anxiolytic effects in female DBA/2JIco mice in the novelty-induced hypophagia (NIH) paradigm. PQ01 reduces anxiety-related behavior in the NIH test. The latency to consume sweetened condensed milk in the home cage and the novel cage as well as the difference in latency is shown. *P<0.05, Mann-Whitney U test. N=13-15 per group.

FIG. 10: Effects of PQ01 in SMAD-Assay. Determination of Smad-regulated signaling activity in CHO cells stably overexpressing TMEFF2 without (left bars, 1-3) and following stimulation with Activin (50 ng) by means of a Smad-Dual-Luciferase Reporter Assay. Cells were either co-incubated with buffer (no AB) or with anti-TMEFF2 antibodies #001 (PQ01) or #16e.

### DETAILED DESCRIPTION OF THE INVENTION

The present invention generally relates to compounds having antidepressive and/or anxiolytic activity leaving normal behavior of the subject to be treated unaffected in kind. In accordance with present invention, antibodies have been tried to be generated and identified which antagonize the action of TMEFF2 on the Activin signaling pathway in order to find candidate compounds for pharmaceutical applications, in particular in connection with the treatment of affective disorders, such as depression, or anxiety disorders. For this purpose, a multitude of antibodies were raised against TMEFF2, wherein one out of several candidates, in particular mouse monoclonal antibody designated in the context of the present application PQ01 showed particularly high efficiency in antagonizing the activity of TMEFF2 in the Activin signaling pathway and having antidepressive or anxiolytic properties as verified in the well-accepted forced-swim-test (FST) and the novelty-induced hypophagia (NIH) test as described in the appended Examples.

Thus, the present invention relates to an anti-TMEFF2 (transmembrane protein with EGF-like and two follistatin-like domains 2) antibody and equivalent binding molecules, characterized in that the antibody and like compounds have one or more, preferably two, more preferably three and most preferably all four of the following properties.
(a) increases Activin induced Smad-regulated signaling pathway activity in TMEFF2 over-expressing CHO cells, see, e.g., Example 7;
(b) displays anxiolytic properties in the novelty-induced hypophagia (NIH) paradigm test, see, e.g., Example 6;
(c) displays antidepressive properties in the forced swim test (FST), see, e.g., Example 4; and
(d) does not substantially change locomotor activity; see, e.g., Example 5;

As demonstrated in the experiments performed in accordance with the present invention by way Examples with subject antibody designated PQ01 in appropriate mouse models, the compounds disclosed herein have therapeutic utility for affective and anxiety disorders thus making the antibody and like molecules particularly suitable for the treatment of these disorders. Furthermore, they do not display side effects otherwise common to antidepressant and anxiolytic drugs such as inducing hyperactivity, excitability or drowsiness. Accordingly, also the risk of addiction for the novel class of antidepressant and anti-anxiety drug seems to be rather low, if any. Since the assays and mouse models used in accordance with the present invention in the Examples represent typical preclinical tests predictive of corresponding clinical trials it is prudent to expect that the class of compounds disclosed herein have therapeutic activity in human as well.

It is known that by modulating TMEFF2 it is possible to provide means and methods for treating affective disorders. TMEFF2 is involved in two signaling pathways. On the one hand TMEFF2 is involved in the cAMP signaling pathway, and on the other hand in the Activin signaling pathway. Moreover, the findings that TMEFF2 is involved in the Activin signaling pathway support the conclusion that TMEFF2 modulators which reduce the binding between Activin and TMEFF2 can be used in the treatment of affective disorders. Thus, a TMEFF2 modulator which reduces the binding of Activin to TMEFF2 enhances the binding of Activin to its receptors which in turn leads to a more efficient activation of the Activin signaling pathway.

Without being bound by theory, it is assumed that the mentioned biological properties are based on or associated with the antibody molecule's capacity of inhibiting the binding of Activin to TMEFF2 thereby allowing Activin to bind to its receptors and to trigger the anti-depressive effects by the Activin signaling pathway. Thus, in one embodiment the antibody molecule according to the present invention is capable of reducing the binding of Activin to TMEFF2. Methods for testing and measuring the capacity of an antibody molecule according to the invention to reduce the binding of Activin to TMEFF2 are known in the art; see, e.g., international application WO2007/090631, the disclosure content of which is incorporated herein by reference.

TMEFF2 modulators including antibodies capable of reducing the binding between Activin and TMEFF2 for the treatment of affective disorders have been contemplated in international application WO2007/090631. However, while this application describes the target signaling pathway and methods how to identify modulators of TMEFF2, proof of concept has been provided with an entirely different approach, i.e. siRNA in order to reduce expression and thus the amount of TMEFF2 in the cell so that Activin may be free to exert its effects on other proteins. A particular antibody which shows the same effect both on the cellular level as well as in the animal model used in WO2007/090631 has not been described.

More importantly, it was not known, let alone could be expected that a TMEFF2 binding molecule such as an antibody could be therapeutically effective in the treatment of both depression and anxiety, and that such antibody does not unspecifically affect general behavior, which is often a problem encountered by antidepressant and anxiolytic drugs available so far. Hence, the present invention for the first time enables the provision of such an antibody and reliable means for its recombinant production, in particular the amino acid sequences of the antibody's variable light and heavy chains including the complementarity determining regions (CDRs).

Hitherto no reliable source of an antibody according to the present invention was available, for example because of loss of specificity or change of specificity of a monoclonal antibody due to the hybridoma cell line producing the initial antibody not being derived from a single cell line. Thus, a mixture of two or more different hybridomas will produce a mixture of two or more monoclonal antibodies with different specificity. The ratio of the cells within the culture and thereby the ratio of the different monoclonal antibodies produced by them may vary during cultivation. Also, a particular hybridoma producing the antibody with the desired specificity might be lost from the mixture if the other hybridoma cells have an evolutionary advantage. A mixture of hybridomas in a culture can also result from mutations in certain cells leading to shifting specificities of the antibodies produced.

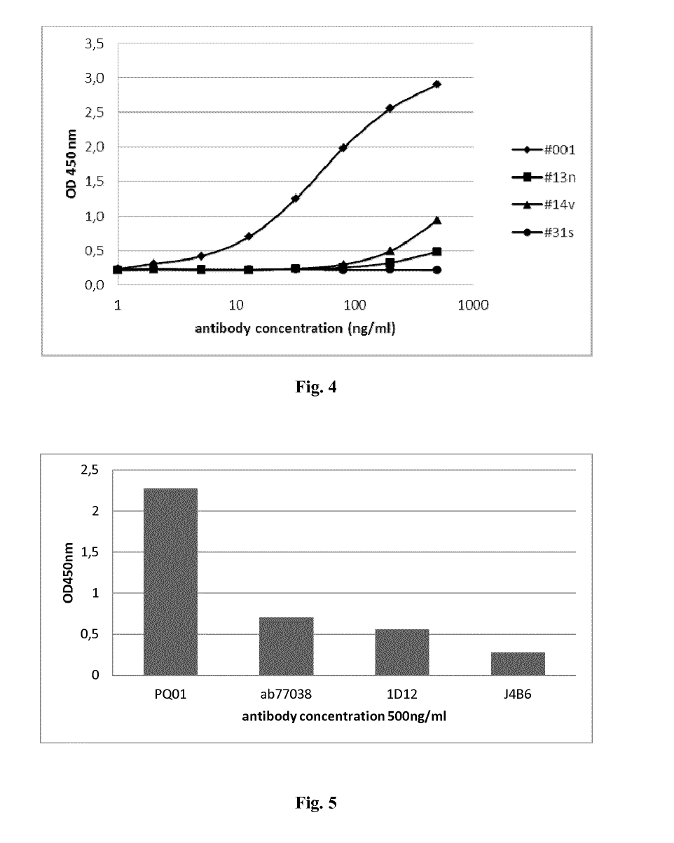

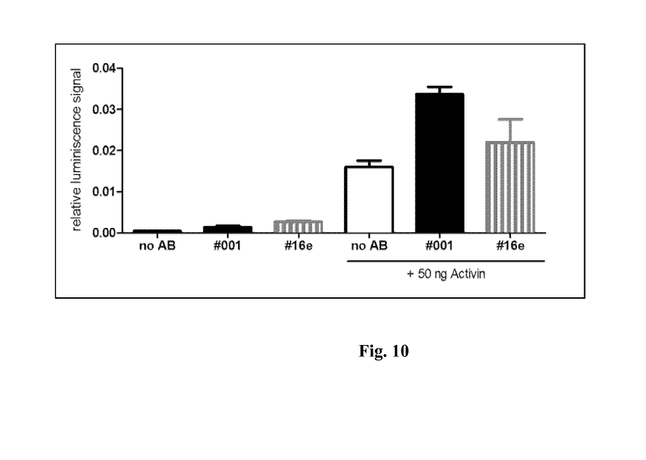

In addition, the antigen used for immunization and generation of the monoclonal antibodies including inter alia monoclonal antibody PQ01 does not seem to be specifically immunogenic. For example, most of the hybridoma subclones obtained after the initial screening produced antibodies with low affinity (see exemplary antibodies PQ10o and PQ13n in FIGS. 3 and 4) and/or having biological activities not as profound as could be demonstrated for PQ01 (see FIG. 10 for antibody clone PQ16e which also in the mouse models proved to be inferior compared to PQ01). The probably low immunogenicity of the antigen and in particular of the epitope recognized by PQ01 is also corroborated by the fact that the commercially available antibody 1D12 from Abnova has been raised against an antigen comprising the epitope recognized by PQ01; see Example 2 and SEQ ID NO: 20. However, as shown in FIG. 5, the affinity of antibody 1D12 is substantially lower than observed for the PQ01 antibody of the present invention.

Thus, it appears as if it was a mere stroke of luck to arrive at the PQ01 antibody since it could and cannot be expected to arrive at such antibody twice if it is tried to raise monoclonal antibodies against the 97aa antigen again. This is all the more true in view of the fact that the monoclonal antibodies obtained with this particular antigen though having equally high binding affinity, i.e. PQ16e, they were significantly different in nature regarding their properties in cell based assays and in particular in the animal models for depression.

This confirms the experience often encountered that knowledge of a target protein or even of an antigen which is more circumscribed used to generate a monoclonal antibody is insufficient for making the original antibody available, though suitable in vitro test systems for screening might be available. Hence, the provision of the amino acid sequences of the variable light and heavy chains of the antibody of the present invention for the first time enables the person skilled in the art to design and produce functionally equivalent antibodies and TMEFF2 binding molecules, for example by adapting the antigen-binding site of antibody PQ01, which fulfill one or more of the following properties.
(a) increases Activin induced Smad-regulated signaling pathway activity in TMEFF2 over-expressing CHO cells, see, e.g., Example 7;
(b) displays anxiolytic properties in the novelty-induced hypophagia (NIH) paradigm test, see, e.g., Example 6;
(c) displays antidepressive properties in the forced swim test (FST), see, e.g., Example 4; and
(d) does not substantially change locomotor activity; see, e.g., Example 5;

The term “TMEFF2” as used in accordance with the present invention denotes a transmembrane protein with EGF-like and two follistatin-like domains 2 (TMEFF2 protein also known as tomoregulin, TR, hyperplastic polyposis gene 1, HPP 1, and TENB2) polypeptide having an amino acid sequence as is known in the art or having an amino acid sequence encoded by a nucleotide sequence as known in the art; see Uchida et al., Biochem. Biophys. Res. Commun. 602 (1999), 266:593; Horie et al., Genomics 67 (2000), 146-152); or Genbank Accession numbers NM_{—}016192, NM_{—}019790, BC034850, BC008973, AY412287, AY412288, AY412289, AB017270, and AB017269. The nucleotide and amino acid sequence of human TMEFF2 as described in Uchida et al. (1999) is also depicted in FIG. 16 of international application WO2007/090631, the disclosure content of which is incorporated herein by reference. When referring herein to TMEFF2, the sequences described in Uchida et al. (1999) and in FIG. 16 of international application WO2007/090631, which presented in SEQ ID NO.: 1 are preferred as “reference sequences” when, e.g. determining the degree of identity of nucleotide or amino acid sequences which are encompassed by the term “TMEFF2”.

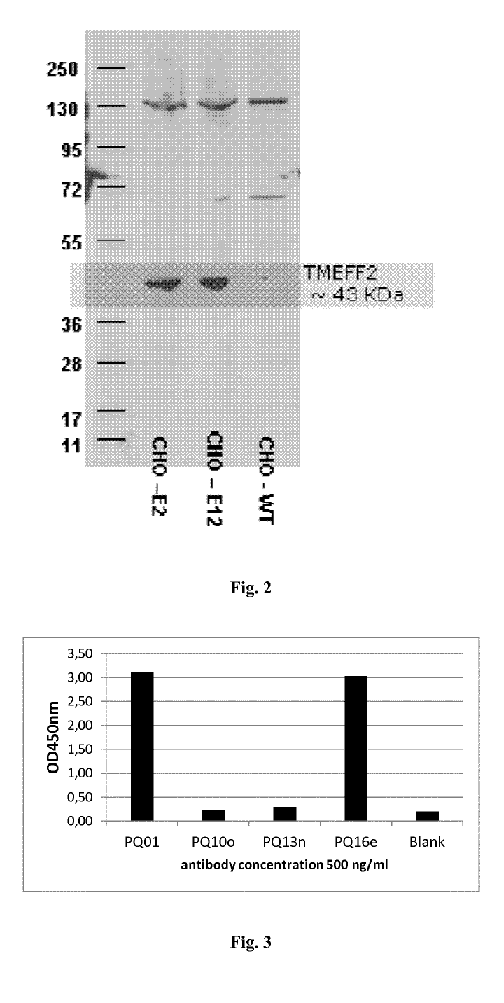

Characterization of their binding specificities towards full-length TMEFF2 on Western Blot (FIG. 2) or by ELISA (FIG. 5) and to the TMEFF2 amino acid fragment 166-262 (FIG. 4) by ELISA confirmed that for the first time monoclonal antibodies have been cloned that are highly specific for human TMEFF2. In one embodiment according to the present invention, the antibody of the present invention is capable of binding TMEFF2 at a concentration of <10 ng/ml, preferably 2 ng/ml. In one embodiment the antibody is capable of binding full length TMEFF2 as determined by ELISA in Example 2 and FIG. 5 showing at OD450 nm an extension of at least 2.0 and/or a at least a threefold increased binding capacity compared to the commercially available antibody 1D12 of Abnova.

The term “Activin”, includes an Activin (also known as inhibin beta A; Activin A; Activin AB alpha polypeptide) polypeptide having an amino acid sequence as known in the art or having an amino acid sequence encoded by a nucleotide sequence as known in the art; see, e.g., Risbridger et al., Endocr. Rev. 22 (2001), 836-858 and FIG. 22 of international application WO2007/090631, the disclosure content of which is incorporated herein by reference

The term “Smad” or “SMAD” includes Smad 2, 3, and 4. In this context, Smad 2 also known as MOTHERS AGAINST DECAPENTAPLEGIC, DROSOPHILA, HOMOLOG OF, 2; SMAD2, MADH2 SMA- AND MAD-RELATED PROTEIN 2 MAD, DROSOPHILA, HOMOLOG OF MADR2V18; MADH2; MADR2; JV18-1; hMAD-2; hSMAD2 is known in the art or Gene Accession number UniProtKB/Swiss-Prot Q15796, HGNC: 6768 Entrez Gene: 4087 Ensembl: ENSG00000175387 OMIM: 601366 and Smad 3 is also known as MOTHERS AGAINST DECAPENTAPLEGIC HOMOLOG 3; MAD homolog 3; Mad3; Mothers against DPP homolog 3; hMAD-3; JV15-2; SMAD family member 3; SMAD 3; UniProtKB/Swiss-Prot: P84022.1, A8K4B6, B7Z4Z5, B7Z9Q2, O09064, O09144, O14510, O35273, Q92940, Q93002, Q9GKR4 BAA22032. Furthermore, Smad4 also known as SMAD, MOTHERS AGAINST DPP HOMOLOG 4 (Drosophila), isoform CRA_a JIP; DPC4; MADH4; MYHRS, is known in the art or Gene ID: 4089, Ensembl:ENSG00000141646; HPRD:02995; MIM:600993; Vega:OTTHUMG00000132696 Accession: EAW62987.1 GI: 119583391.

The increase of Activin induced Smad-regulated signaling pathway activity in TMEFF2 over-expressing mammalian cells such as CHO cells can be determined by using a functional assay. This is a preferred method in order to determine the capacity of an antibody according to the invention to interfere of the binding of TMEFF2 and Activin. Free Activin binds to the Activin receptors and this results in receptor activation, phosphorylation, and Smad activation. Therefore, the reduced binding between TMEFF2 and Activin can be measured by an increase of receptor or Smad phosphorylation as well as an increase of Smad transcriptional activity. The Smad transcriptional activity can be measured for example with a reporter construct having a sequence 12XCAGA cloned in the enhancer region of a Luciferase reporter.

A corresponding assay is described in the appended Example 7 and shown in FIG. 10. It is preferred that in an assay as described in Example 7, an antibody according to the present invention leads to an increase in relative luminescence of at least 50%, preferably of at least 60%, 70%, 80% or 90%, most preferably of an increase of at least 100% when compared to the control, i.e. the activation with Activin without addition of an anti-TMEFF2 antibody. In a particularly preferred embodiment, the antibody according to the present invention shows in the assay as described in Example 7 an increase in relative luminescence which is at least as high as the increase observed with the antibody PQ01.

As mentioned hereinabove and demonstrated in the examples the compounds disclosed herein have therapeutic utility for affective and/or anxiety disorders, thus making the antibody and like molecules particularly suitable for the treatment of these disorders. The term “affective disorder” is used in this context according to the ICD-10 (30-F39), supra. Accordingly, affective disorders comprise depressive episode, recurrent depressive disorder, manic episode, bipolar affective disorder, persistent mood disorders, other mood disorders, unspecified mood disorder.

The term “anxiety disorder” is used in this context according to the ICD-10 (F40-F48), supra. Accordingly, anxiety disorder in particular relates to phobic anxiety disorders, other anxiety disorders, obsessive-compulsive disorder, reaction to severe stress and adjustment disorders, dissociative disorders, somatoform disorders, other neurotic disorders. Preferably, the term “anxiety disorder” relates to acute stress disorder, generalized anxiety disorder, and posttraumatic stress disorder.

The term “anxiolytic properties” means that the antibody molecule qualifies in an accepted test as an anxiolytic compound. Such a test is the novelty-induced hypophagia (NIH) paradigm. This test is described in the appended Example 6 and FIG. 9. Preferably, an anxiolytic activity is acknowledged if latency to consume the palatable fluid in the novel cage is significantly decreased when compared to the vehicle control group. Preferably, “significantly decreased” means that the latency to consume the palatable fluid in the novel cage is decreased by at least 10%, preferably by at least 20%, and even more preferably by at least 30% when compared to the vehicle control group.

The term “depressive disorder” in this context preferably refers to a major depressive disorder (single episode or recurrent), dysthymic disorder or depressive disorder NOS (not otherwise specified).

The term “antidepressive properties” means that the antibody molecule qualifies in an accepted test as an antidepressant compound. Such a test is the forced swim test (FST) paradigm. This test is described in the appended Example 4 and FIG. 7. Preferably, an antidepressive effect is acknowledged if the active escape behavior (i.e. time struggling) is significantly increased when compared to the vehicle control group. Preferably, “significantly increased” means that the time of struggling is increased by at least 10%, preferably by at least 20% and even more preferably by at least 30% when compared to the control. Moreover, preferably an antidepressant effect is acknowledged if the passive stress coping behavior (i.e. time floating) is significantly decreased when compared to the vehicle control group. Preferably, “significantly decreased” means that the time of floating is decreased by at least 10%, preferably by at least 20%, and even more preferably by at least 30% when compared to the control.

The term “bipolar disorder” preferably comprises bipolar I disorder, bipolar II disorder, cyclothymic disorder, and bipolar disorder NOS.

In a preferred embodiment of the present invention, the antibody or equivalent binding molecule recognizes a unique epitope that is contained within and essentially consists of, respectively, the amino acid sequence EDGHYAR (SEQ ID NO:13) of TMEFF2. Most preferably, said antibody is a monoclonal antibody.

In particular, antibodies and antigen-binding fragments thereof are provided, which demonstrate the immunological binding characteristics and/or biological properties as outlined for the antibody illustrated below and in the Examples. Where present, the term “immunological binding characteristics,” or other binding characteristics of an antibody with an antigen, in all of its grammatical forms, refers to the specificity, affinity, cross-reactivity, and other binding characteristics of an antibody.

Thus, the present invention is directed to an anti-TMEFF2 antibody, or antigen-binding fragment, variant or derivatives thereof, wherein the antibody specifically binds to the same epitope of TMEFF2 as a reference antibody PQ01. As illustrated in the Examples, antibodies generated in accordance with the present invention recognized the wild type TMEFF2, i.e. a 43kD band in TMEFF2 overexpressing cells corresponding to the size for the TMEFF2 protein. Furthermore, antibody PQ01 indicated as #001 or PQ001 binds to the 97 amino acid fragment of TMEFF2 (166-262) or to human full length; in a direct ELISA assay; see FIGS. 4 and 5, respectively. In addition, the signals obtained with various TMEFF2 antibodies (#001, #13n, #14v, #31s. #16e) produced as described in Example 1 are not only concentration-dependent but also reflect sub-clone specific binding properties with the antibody designated PQ01 (#001) showing binding already at a concentration at 2 ng/ml; see FIG. 4. In addition, several antibodies directed against the TMEFF2 are commercially available for example from Abnova (1D12) or Abcam (ab77038). However, in accordance with the experiments as outlined in Example 2 and FIG. 5 none of these showed at an antibody concentration of 500 ng/ml at OD 450 nm a binding to the commercially available full length TMEFF2 protein (Abnova) as the antibody of the present invention, which exhibit a threefold higher binding capacity than the 1D12 in ELISA binding assay.

Thus, in one embodiment the antibody is capable of binding full length TMEFF2 as determined by ELISA in Example 2 FIG. 5 showing at OD450 nm an extension of at least, 1.5, preferable 2, preferably 2.1 and/or a at least 1.5, preferably, twofold, preferably threefold increased binding capacity compared to the commercially available antibody 1D12 of Abnova.

Also, the present invention provides the binding of an antibody molecule to TMEFF2 as assessed in an ELISA assay, more preferably in an ELISA assay as described in Example 2. In a particularly preferred embodiment the antibody molecule of the present invention, when tested in the ELISA assay as described in Example 2, is capable of binding to TMEFF2 at a concentration of 100 ng/ml or less, more preferably of 50 ng/ml or less, of 20 ng/ml or less, of 10 ng/ml or less, most preferably of 5 ng/ml of less or of 3 ng/ml or less, and in particular at a concentration of 2 ng/ml. In a preferred embodiment of the present invention, the anti-TMEFF2 antibody is capable of binding TMEFF2 at a concentration of <10 ng/ml, preferably 2 ng/ml.

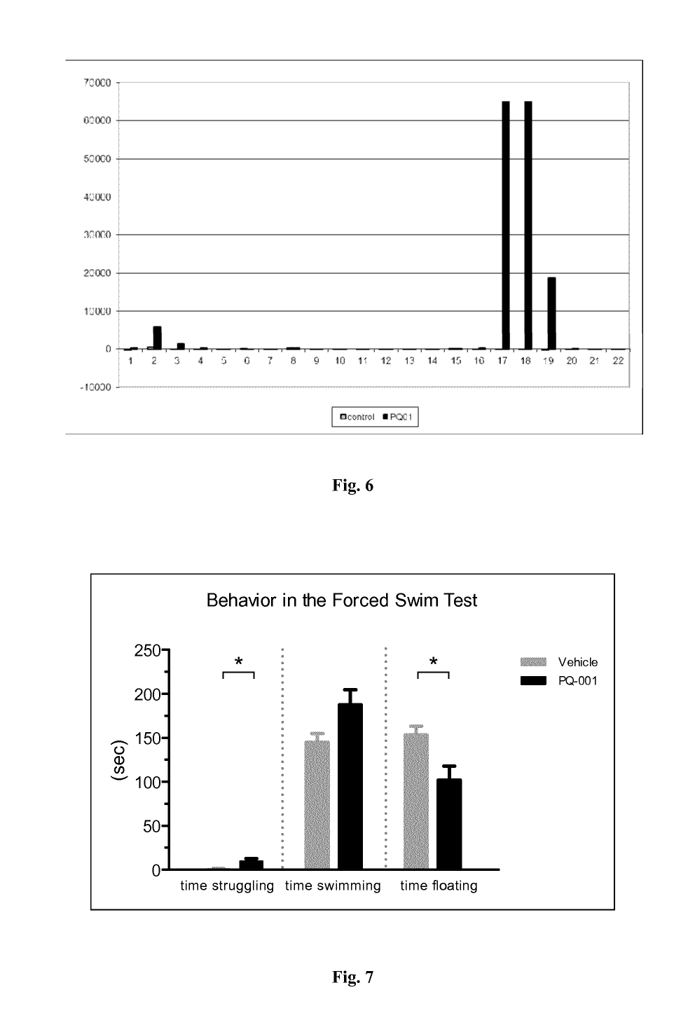

Further, without intending to be bound by initial experiment observations as demonstrated in Example 3 the monoclonal PQ01 anti-TMEFF2 antibody of the present invention is preferably characterized in significant binding to an epitope contained in peptides 17, 18, and 19. The antibody molecule according to the present invention is furthermore characterized in that it binds to an epitope comprising the amino acid sequence EDGHYAR (SEQ ID NO: 13) which corresponds to residues 238 to 244 of the amino acid sequence of human TMEFF2. Peptide mapping of the antigen recognized by the antibody PQ01 revealed that the antibody binds to a region of human TMEFF2 which contains this sequence see also FIG. 6. In particular, the antibody PQ01 is capable of binding to three peptides (shown in SEQ ID NOs: 14 to 16) each of which contains this sequence. The overlapping sequence in these peptides is EDGHYAR (SEQ ID NO: 13) and the overall sequence represented by peptides 17, 18, and 19 is NTTTTTKSEDGHYARTDYAENAN (SEQ ID NO:17). Thus in one embodiment of the present invention the anti-TMEFF2 antibody or binding fragment thereof is capable of binding an epitope comprising the amino acid sequence EDGHYAR (SEQ ID NO: 13). In a preferred embodiment of the present invention the antibody or antigen-binding fragment thereof is capable of binding a peptide consisting of the amino acid sequence NTTTTTKSEDGHYAR (SEQ ID NO: 14), a peptide consisting of the amino acid sequence TTKSEDGHYARTDYA (SEQ ID NO: 15), and/or a peptide consisting of EDGHYARTDYAENAN (SEQ ID NO: 16)

The binding of the antibody molecule to an epitope containing the amino acid sequence EDGHYAR (SEQ ID NO: 13) or to one of the above-mentioned peptides can, for example, be verified by immunostaining or immunoisolation. Methods for immunostaining are well-known in the art. Non-limiting examples for immunostaining are immunohistochemistry, immunocytochemistry (Spector and Goldmann Cells: A laboratory manual, vol. 2 (1998): Light microscopy and cell structure), flow cytometry (Ormerod, Flow Cytometry: A Practical Approach, 3rd edition (2000); or Nebe-von-Caron et al., J. Microbiol. Methods (2000); 42:97-114), ELISA (Engvall and Perlman, Immunochemistry 8 (1971), 871-874; Goldsby et al., Enzyme-Linked Immunosorbent Assay: Immunology, 5th edition (2003), 148-150. W. H. Freeman, New York, 2003) and/or immunoelectron microscopy (Matutes and Catovsky, Clin. Exp. Immunol. 50 (1982), 416-425). Non-limiting examples for immunoisolation are immunoprecipitation (Kessler, J. Immunol. 115 (1975), 1617-1623), immuno affinity purification (Gersten and Marchalonis, J. Immunol. Methods 24 (1978), 305-309), and/or ELISA (Butler, Mol. Immunol. 23 (1986), 971-982; Tanaka et al., J. Agric. Food Chem. 55 (2007), 3783-3787; Renart et al., Proc. Natl. Acad. Sci. U.S.A. 76 (1979), 3116-3120; Towbin et al., Proc. Natl. Acad. Sci. U.S.A. 76 (1979), 4350-4354; Burnette, Anal. Biochem. 112 (1981), 195-203).

One example of a binding assay which is suitable for verifying the binding of an antibody molecule according to the invention to an epitope or a peptide comprising the amino acid sequence EDGHYAR (SEQ ID NO: 13) is described in Example 3 and depicted in FIG. 6.

Naturally, the present invention extends to the antibody producing cell lines and recombinant cells as well. Thus, the present invention advantageously provides recombinant means and indefinitely prolonged cells as a source of a monoclonal antibody of the present invention. The present invention further relates to diagnostic assays and kits that comprise the antibody of the present invention or an equivalent binding molecule and to therapeutic methods based thereon.

Thus, the present invention generally relates to any antibody, in particular monoclonal antibody, antigen-binding fragments thereof and equivalent binding molecules which demonstrate the immunological binding characteristics and preferably biological activity of the PQ01 antibody of the present invention as described above and demonstrated in the Examples.

A “binding molecule” as used in the context of the present invention relates primarily to antibodies, and fragments thereof, but may also refer to other non-antibody molecules that bind to TMEFF2 and exhibit the functional properties of the PQ01 antibody of the present invention including but not limited to hormones, receptors, ligands, major histocompatibility complex (MHC) molecules, chaperones such as heat shock proteins (HSPs) as well as cell-cell adhesion molecules such as members of the cadherin, intergrin, C-type lectin, immunoglobulin (Ig) superfamilies and in particular designed ankyrin repeat proteins (DARPins) which are a promising class of non-immunoglobulin proteins that can offer advantages over antibodies for target binding; see for review, e.g., Stumpp and Amstutz, Curr. Opin. Drug Discov. Devel. 10 (2007), 153-159, and references cited therein. Thus, for the sake of clarity only and without restricting the scope of the present invention most of the following embodiments are discussed with respect to antibodies and antibody-like molecules which represent the preferred binding molecules for the development of therapeutic and diagnostic agents. Antibodies or antigen-binding fragments, immunospecific fragments, variants, or derivatives thereof of the invention include, but are not limited to, polyclonal, monoclonal, multispecific, murine, human, humanized, primatized, murinized or chimeric antibodies, a recombinant full antibody (immunoglobulin), in particular a monoclonal recombinant full antibody (immunoglobulin), single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library, and anti-idiotypic (anti-Id) antibodies (including, e.g., anti-Id antibodies to antibodies disclosed herein), a chimeric antibody, a CDR-grafted antibody, a bivalent antibody-construct, a synthetic antibody, a cross-cloned antibody, a fully-human antibody, a humanized antibody, nanobodies, diabodies, and the like. ScFv molecules are known in the art and are described, e.g., in U.S. Pat. No. 5,892,019. Immunoglobulin or antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Means and methods for the recombinant production of binding molecules, in particular antibodies and mimics thereof as well as methods of screening for competing binding molecules, which may or may not be antibodies, are known in the art and are summarized, for example, in international application WO2006/103116 with respect to antibodies against beta-amyloid and the treatment/diagnosis of Alzheimer's disease, the disclosure content of which is incorporated herein by reference for this purpose of antibody engineering and administration for therapeutic or diagnostic applications.

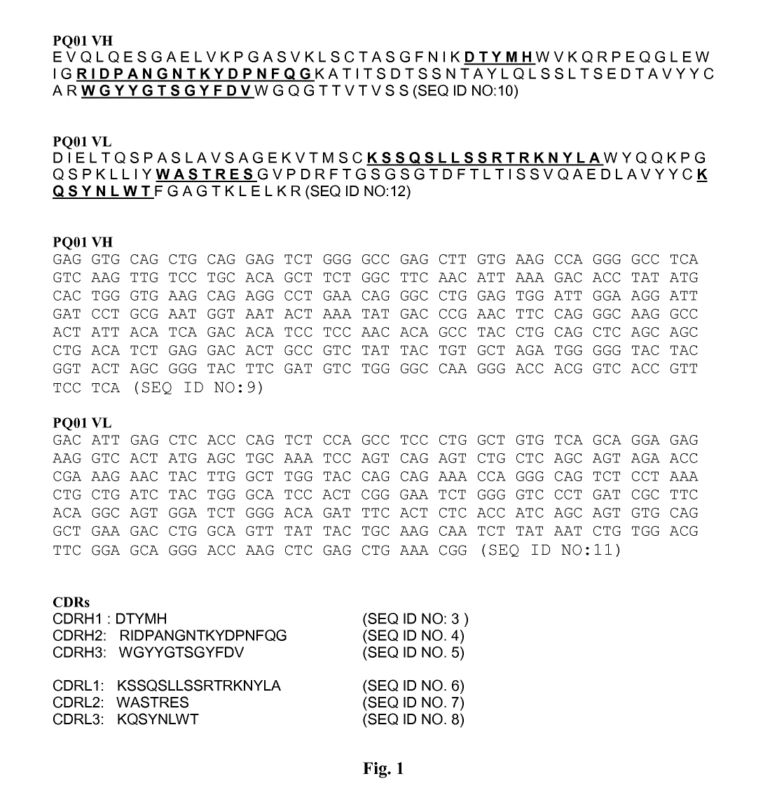

Typically, the antibody of the present invention comprises in its epitope binding domain, i.e. variable region (a) at least one complementarity determining region (CDR) of the VH and/or VL variable region amino acid sequences depicted in (i) FIG. 1 (VH) (SEQ ID NOs: 3, 4, 5); and (ii) FIG. 1 (VL) (SEQ ID NOs: 6, 7, 8); (b) an amino acid sequence of the VH and/or VL region as depicted in FIG. 1; (c) at least one CDR consisting of an amino acid sequence resulted from a partial alteration of any one of the amino acid sequences of (a); (d) a heavy chain and/or light variable region comprising an amino acid sequence resulted from a partial alteration of the amino acid sequence of (b); or (e) at least one CDR comprising an amino acid sequence with at least 90% identity to any one of the amino acid sequences of (a).

In a particularly preferred embodiment of the present invention, the human antibody or antigen-binding fragment thereof comprising H-CDRs 1 to 3 taken as a whole which are at least 95% identical to SEQ ID NOs: 3, 4, 5 taken as a whole and L-CDRs 1 to 3 taken as a whole which are at least 95% identical to SEQ ID NOs: 6, 7, 8 taken as a whole.

In one embodiment, the antibody of the present invention is any one of antibody comprising an amino acid sequence of the VH and/or VL region as depicted in FIG. 1. Alternatively, the antibody of the present invention is an antibody or antigen-binding fragment thereof, which competes for binding to the TMEFF2 with the antibody having the VH and VL region as depicted in FIG. 1. Those antibodies may be murine, however, humanized, xenogeneic, or chimeric human-murine antibodies being preferred, in particular for therapeutic applications. However, for diagnostic uses and research in general murine antibodies are suitable as well. An antigen-binding fragment of the antibody can be, for example, a single chain Fv fragment (scFv), a F(ab′) fragment, a F(ab) fragment, and an F(ab′)₂ fragment.

Competition between antibodies is determined by an assay in which the immunoglobulin under test inhibits specific binding of a reference antibody to a common antigen, such as TMEFF2. Numerous types of competitive binding assays are known, for example: solid phase direct or indirect radioimmunoassay (RIA), solid phase direct or indirect enzyme immunoassay (EIA), sandwich competition assay; see Stahli et al., Methods Enzymol. 9 (1983), 242-253; solid phase direct biotin-avidin EIA; see Kirkland et al., J. Immunol. 137 (1986), 3614-3619 and Cheung et al., Virology 176 (1990), 546-552; solid phase direct labeled assay, solid phase direct labeled sandwich assay; see Harlow and Lane, Antibodies, A Laboratory Manual, Cold Spring Harbor Press (1988); solid phase direct label RIA using 1125 label; see Morel et al, Molec. Immunol. 25 (1988), 7-15, and Moldenhauer et al., Scand. J. Immunol. 32 (1990), 77-82. Typically, such an assay involves the use of purified TMEFF2 or aggregates thereof bound to a solid surface or cells bearing either of these, an unlabelled test immunoglobulin and a labeled reference immunoglobulin, i.e. the monoclonal antibody of the present invention. Competitive inhibition is measured by determining the amount of label bound to the solid surface or cells in the presence of the test immunoglobulin. Usually the test immunoglobulin is present in excess. Preferably, the competitive binding assay is performed under conditions as described for the ELISA assay in the appended Examples. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Usually, when a competing antibody is present in excess, it will inhibit specific binding of a reference antibody to a common antigen by at least 50% or 75%. Hence, the present invention is further drawn to an antibody, or antigen-binding fragment, variant or derivatives thereof capable of inhibiting a reference antibody PQ01 from binding to TMEFF2 and/or competing with its binding.

For some applications only the variable regions of the antibodies are required, which can be obtained by treating the antibody with suitable reagents so as to generate Fab′, Fab, or F(ab″)₂ portions. Such fragments are sufficient for use, for example, in immunodiagnostic procedures involving coupling the immunospecific portions of immunoglobulins to detecting reagents such as radioisotopes.

In accordance with the above, the present invention also relates to a polynucleotide encoding the binding molecule of the present invention, in case of the antibody preferably at least the binding domain or variable region of an immunoglobulin chain of the antibody described above. Typically, said variable region encoded by the polynucleotide comprises at least one complementarity determining region (CDR) of the VH and/or VL of the variable region of the said antibody. The person skilled in the art knows that each variable domain (the heavy chain VH and light chain VL) of an antibody comprises three hypervariable regions, sometimes called complementarity determining regions or “CDRs” flanked by four relatively conserved framework regions or “FRs” and refer to the amino acid residues of an antibody which are responsible for antigen-binding. The hypervariable regions or CDRs of the human IgG subtype of antibody comprise amino acid residues from residues 24-34 (L1), 50-56 (L2) and 89-97 (L3) in the light chain variable domain and 31-35 (H1), 50-65 (H2) and 95-102 (H3) in the heavy chain variable domain as described by Kabat et al., Sequences of Proteins of Immunological Interest, 5th Ed Public Health Service, National Institutes of Health, Bethesda, Md. (1991) and/or those residues from a hypervariable loop, i.e. residues 26-32 (L1), 50-52 (L2) and 91-96 (L3) in the light chain variable domain and 26-32 (H1), 53-55 (H2) and 96-101 (H3) in the heavy chain variable domain as described by Chothia et al., J. Mol. Biol. 196 (1987), 901-917. Framework or FR residues are those variable domain residues other than and bracketing the hypervariable regions. The term “specific binding” and “high affinity”, respectively, refers to antibody binding to a predetermined antigen, i.e. the TMEFF2 epitope defined above. Typically, the antibody binds with a dissociation constant (K_{D}) of 10⁻⁷ M or less, and binds to the predetermined antigen with a K_{D} that is at least twofold less than its K_{D} for binding to a nonspecific antigen (e.g., BSA, casein, or any other specified polypeptide) other than the predetermined antigen. The phrases “an antibody recognizing an antigen” and “an antibody specific for an antigen” are used interchangeably herein with the term “an antibody which binds specifically to an antigen”. As used herein “highly specific” binding means that the relative K_{D} of the antibody for the specific TMEFF2 epitope is at least 10-fold less than the K_{D} for binding that antibody to other ligands.

The affinity or avidity of an antibody for an antigen can be determined experimentally using any suitable method; see, for example, Berzofsky et al., Antibody-Antigen Interactions, Fundamental Immunology, Paul, W. E., Ed., Raven Press New York, N Y (1984), Kuby, Janis Immunology, W. H. Freeman and Company New York, N Y (1992), and methods described herein. The measured affinity of a particular antibody-antigen interaction can vary if measured under different conditions, e.g., salt concentration, pH. Thus, measurements of affinity and other antigen-binding parameters, e.g., K sub D, IC50, are preferably made with standardized solutions of antibody and antigen, and a standardized buffer.

The person skilled in the art will readily appreciate that the variable domain of the antibody having the above-described variable domain can be used for the construction of other polypeptides or antibodies of desired specificity and biological function. Thus, the present invention also encompasses polypeptides and antibodies comprising at least one CDR of the above-described variable domain and which advantageously have substantially the same or similar binding properties as the antibody described in the appended Examples. The person skilled in the art will readily appreciate that using the variable domains or CDRs described herein antibodies can be constructed according to methods known in the art, e.g., as described in European patent applications EP 0 451 216 A1 and EP 0 549 581 A1. Furthermore, the person skilled in the art knows that binding affinity may be enhanced by making amino acid substitutions within the CDRs or within the hypervariable loops (Chothia and Lesk, J. Mol. Biol. 196 (1987), 901-917) which partially overlap with the CDRs as defined by Kabat. Thus, the present invention also relates to antibodies wherein one or more of the mentioned CDRs comprise one or more, preferably not more than two or three amino acid substitutions or even more amino acids in case of CDR2 and CDR3. Preferably, the antibody of the invention comprises in one or both of its immunoglobulin chains two or all three CDRs of the variable regions as set forth in FIG. 1.

In a preferred embodiment the antibody is a human, humanized or a synthetic human antibody. Preferably, said antibody is a human chimeric, humanized or fully human antibody, for example in order to avoid the development of Human Anti-Mouse Antibodies (HAMA) response in a human subject; see also infra. The chimeric antibodies can comprise portions derived from two different species (e.g., human constant region and murine variable or binding region). The portions derived from two different species can be joined together chemically by conventional techniques or can be prepared as single contiguous proteins using genetic engineering techniques. DNA encoding the proteins of both the light chain and heavy chain portions of the chimeric antibody can be expressed as contiguous proteins.

As used herein, the term “humanized” or “humanization” are used interchangeably to refer to an antibody comprising in its binding domains at least one complementarity determining region (CDR) from a non-human antibody or fragment thereof. Humanization approaches are described for example in WO 91/09968 and U.S. Pat. No. 6,407,213; see also supra. As non-limiting examples, the term encompasses the case in which a variable region of the binding domain comprises a single CDR region from another non-human animal, for example a rodent, as well as the case in which a or both variable region/s comprise at each of their respective first, second and third CDRs the CDRs from said non-human animal. Optionally, the framework region of the monoclonal antibody is aligned and adopted in accordance with the pertinent human germ line variable region sequences in the database; see, e.g., Vbase (http://vbase.mrc-cpe.cam.ac.uk/) hosted by the MRC Centre for Protein Engineering (Cambridge, UK). For example, amino acids considered to deviate from the human germ line sequence could be replaced with the corresponding amino acid in the human framework sequence.

In addition, further modifications can be applied to the antibody or binding molecule of the present invention in order to improve the delivery of the antibody to its target site within the patient. Thus, in one embodiment of the present invention the antibody, or antigen-binding fragment, or the antigen-binding molecule as described above further comprising a penetration enhancing peptide.

Use of a penetration enhancing peptide (CCP) improves the delivery of the drug molecule such as antibody or antigen-binding molecule by increasing the rate and extent of transport of the delivery a molecule into the cells. CPPs typically have an amino acid composition that either contains 6 to 8 polycationic or amphipathic amino acids such as peptide LVGVFH or otherwise known in the art, see Wagstaff et al., Curr. Med. Chem. 13 (2006), 1371-1387 the disclosure content is incorporated herein by reference. Such a delivery system based on compositions of liposomes to deliver molecules, antibodies, drugs or genes to the brain that are comparable to the viral vectors, yet overcome the immunogenicity issues. This system is unique in the fact that it is a combination of targeting polypeptides and cell penetrating peptide in one single delivery system.

The polynucleotide of the invention encoding the above described antibody may be, e.g., DNA, cDNA, RNA or synthetically produced DNA or RNA or a recombinantly produced chimeric nucleic acid molecule comprising any of those polynucleotides either alone or in combination. Preferably said polynucleotide is part of a vector. Such vectors may comprise further genes such as marker genes which allow for the selection of said vector in a suitable host cell and under suitable conditions.

Preferably, the polynucleotide of the invention is operatively linked to expression control sequences allowing expression in prokaryotic or eukaryotic cells. Expression of said polynucleotide comprises transcription of the polynucleotide into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers, and/or naturally associated or heterologous promoter regions.

In this respect, the person skilled in the art will readily appreciate that the polynucleotides encoding at least the variable domain of the light and/or heavy chain may encode the variable domains of both immunoglobulin chains or only one. Likewise, said polynucleotides may be under the control of the same promoter or may be separately controlled for expression. Possible regulatory elements permitting expression in prokaryotic host cells comprise, e.g., the P_{L}, lac, trp or tac promoter in E. coli, and examples for regulatory elements permitting expression in eukaryotic host cells are the AOX1 or GAL1 promoter in yeast or the CMV-, SV40-, RSV-promoter, CMV-enhancer, SV40-enhancer or a globin intron in mammalian and other animal cells.

Beside elements which are responsible for the initiation of transcription such regulatory elements may also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium may be added to the coding sequence of the polynucleotide of the invention and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including a C- or N-terminal identification peptide imparting desired characteristics, e.g., stabilization or simplified purification of expressed recombinant product. In this context, suitable expression vectors are known in the art such as Okayama-Berg cDNA expression vector pcDV1 (Pharmacia), pCDM8, pRc/CMV, pcDNA1, pcDNA3 (Invitrogen), or pSPORT1 (GIBCO BRL).

Preferably, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high level expression of the nucleotide sequences, and, as desired, the collection and purification of the immunoglobulin light chains, heavy chains, light/heavy chain dimers or intact antibodies, binding fragments or other immunoglobulin forms may follow; see, Beychok, Cells of Immunoglobulin Synthesis, Academic Press, N.Y., (1979).

Furthermore, the present invention relates to vectors, particularly plasmids, cosmids, viruses, and bacteriophages used conventionally in genetic engineering that comprise a polynucleotide encoding a variable domain of an immunoglobulin chain of an antibody of the invention; optionally in combination with a polynucleotide of the invention that encodes the variable domain of the other immunoglobulin chain of the antibody of the invention. Preferably, said vector is an expression vector and/or a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook et al., Molecular Cloning A Laboratory Manual, Cold Spring Harbor Laboratory N.Y. (1989) and Ausubel, Current Protocols in Molecular Biology, Green Publishing Associates and Wiley Interscience, N.Y. (1994). Alternatively, the polynucleotides and vectors of the invention can be reconstituted into liposomes for delivery to target cells. The vectors containing the polynucleotides of the invention (e.g., the heavy and/or light variable domain(s) of the immunoglobulin chains encoding sequences and expression control sequences) can be transferred into the host cell by well known methods, which vary depending on the type of cellular host. For example, calcium chloride transfection is commonly utilized for prokaryotic cells, whereas calcium phosphate treatment or electroporation may be used for other cellular hosts; see Sambrook, supra.

The present invention furthermore relates to host cells transformed with a polynucleotide or vector of the invention. Said host cell may be a prokaryotic or eukaryotic cell. The polynucleotide or vector of the invention which is present in the host cell may either be integrated into the genome of the host cell or it may be maintained extrachromosomally. The host cell can be any prokaryotic or eukaryotic cell, such as a bacterial, insect, fungal, plant, animal or human cell. Preferred fungal cells are, for example, those of the genus Saccharomyces, in particular those of the species S. cerevisiae. The term “prokaryotic” is meant to include all bacteria which can be transformed or transfected with a DNA or RNA molecules for the expression of an antibody of the invention or the corresponding immunoglobulin chains. Prokaryotic hosts may include gram negative as well as gram positive bacteria such as, for example, E. coli, S. typhimurium, S. marcescens and B. subtilis. The term “eukaryotic” is meant to include yeast, higher plant, insect and preferably mammalian cells, most preferably HEK 293, NSO and CHO cells and derivates thereof like DG44, CHO-K1 and/or other cells like AG8. Depending upon the host employed in a recombinant production procedure, the antibodies or immunoglobulin chains encoded by the polynucleotide of the present invention may be glycosylated or may be non-glycosylated. Antibodies of the invention or the corresponding immunoglobulin chains may also include an initial methionine amino acid residue. A polynucleotide of the invention can be used to transform or transfect the host using any of the techniques commonly known to those of ordinary skill in the art. Furthermore, methods for preparing fused, operably linked genes and expressing them in, e.g., mammalian cells and bacteria are well-known in the art (Sambrook et al., Molecular Cloning: A Laboratory Manual, Cold Spring Harbor Laboratory, Cold Spring Harbor, N.Y., 1989). The genetic constructs and methods described therein can be utilized for expression of the antibody of the invention or the corresponding immunoglobulin chains in eukaryotic or prokaryotic hosts. In general, expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells. Suitable source cells for the DNA sequences and host cells for immunoglobulin expression and secretion can be obtained from a number of sources, such as the American Type Culture Collection (Catalogue of Cell Lines and Hybridomas, 5. eds. (1985) Rockville, Md., U.S.A., which is incorporated herein by reference). Furthermore, transgenic animals, preferably mammals, comprising cells of the invention may be used for the large scale production of the antibody of the invention.

Thus, in a further embodiment, the present invention relates to a method for the production of an antibody or a binding fragment or immunoglobulin chain(s) thereof, said method comprising
(a) culturing a cell as described above; and
(b) isolating said antibody or binding fragment or immunoglobulin chain(s) thereof from the culture.

The transformed hosts can be grown in fermentors and cultured according to techniques known in the art to achieve optimal cell growth. Once expressed, the whole antibodies, their dimers, individual light and heavy chains, or other immunoglobulin forms of the present invention, can be purified according to standard procedures of the art, including ammonium sulfate precipitation, affinity columns, column chromatography, gel electrophoresis, and the like; see, Protein Purification, Springer Verlag, N.Y. (1982). The antibody or its corresponding immunoglobulin chain(s) of the invention can then be isolated from the growth medium, cellular lysates, or cellular membrane fractions. The isolation and purification of the, e.g., recombinantly expressed antibodies or immunoglobulin chains of the invention may be by any conventional means such as, for example, preparative chromatographic separations and immunological separations such as those involving the use of monoclonal or polyclonal antibodies directed, e.g., against the constant region of the antibody of the invention. It will be apparent to those skilled in the art that the antibodies of the invention can be further coupled to other moieties for, e.g., drug targeting and imaging applications. Such coupling may be conducted chemically after expression of the antibody to site of attachment or the coupling product may be engineered into the antibody of the invention at the DNA level. The DNAs are then expressed in a suitable host system, and the expressed proteins are collected and renatured, if necessary.

Substantially pure immunoglobulins of at least about 90 to 95% homogeneity are preferred, and 98 to 99% or more homogeneity most preferred, for pharmaceutical uses. Once purified, partially or to homogeneity as desired, the antibodies may then be used therapeutically (including extracorporally) or in developing and performing assay procedures.

The present invention also involves a method for producing cells capable of expressing an antibody of the invention or its corresponding immunoglobulin chain(s) comprising genetically engineering cells with the polynucleotide or with the vector of the invention. The cells obtainable by the method of the invention can be used, for example, to test the interaction of the antibody of the invention with its antigen.

As mentioned before, the immunoglobulin or its encoding cDNAs may be further modified. Thus, in a further embodiment the method of the present invention comprises any one of the step(s) of producing a chimeric antibody, humanized antibody, single-chain antibody, Fab-fragment, bi-specific antibody, fusion antibody, labeled antibody or an analog of any one of those. Corresponding methods are known to the person skilled in the art and are described, e.g., in Harlow and Lane, Antibodies, A Laboratory Manual, CSH Press, Cold Spring Harbor (1988). When derivatives of said antibodies are obtained by the phage display technique, surface plasmon resonance as employed in the BIAcore system can be used to increase the efficiency of phage antibodies which bind to the same epitope as that of any one of the antibodies described herein (Schier, Hum. Antibodies Hybridomas 7 (1996), 97-105; Malmborg, J. Immunol. Methods 183 (1995), 7-13). The production of chimeric antibodies is described, for example, in international application WO 89/09622. Methods for the production of humanized antibodies are described in, e.g., European application EP-A1 0 239 400 and international application WO 90/07861. A further source of antibodies to be utilized in accordance with the present invention are so-called xenogeneic antibodies. The general principle for the production of xenogeneic antibodies such as human antibodies in mice is described in, e.g., international applications WO 91/10741, WO 94/02602, WO 96/34096, and WO 96/33735. As discussed above, the antibody of the invention may exist in a variety of forms besides complete antibodies; including, for example, Fv, Fab, and F(ab)₂, as well as in single chains; see e.g. international application WO 88/09344. Furthermore, diabodies and V-like domain binding molecules are well-known to the person skilled in the art; see, e.g. U.S. Pat. No. 7,166,697.

The antibodies of the present invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook et al., Molecular Cloning A Laboratory Manual, Cold Spring Harbor Laboratory N.Y. (1989) and Ausubel, Current Protocols in Molecular Biology, Green Publishing Associates and Wiley Interscience, N.Y. (1994). Modifications of the antibody of the invention include chemical and/or enzymatic derivatizations at one or more constituent amino acids, including side chain modifications, backbone modifications, and N- and C-terminal modifications including acetylation, hydroxylation, methylation, amidation, and the attachment or removal of carbohydrate or lipid moieties, cofactors, and the like. Likewise, the present invention encompasses the production of chimeric proteins which comprise the described antibody or some fragment thereof at the amino terminus fused to heterologous molecule such as an immunostimulatory ligand at the carboxyl terminus; see, e.g., international application WO 00/30680 for corresponding technical details.

Additionally, the present invention encompasses small peptides including those containing a binding molecule as described above, for example containing the CDR3 region of the variable region of any one of the mentioned antibodies, in particular CDR3 of the heavy chain since it has frequently been observed that heavy chain CDR3 (HCDR3) is the region having a greater degree of variability and a predominant participation in antigen-antibody interaction. Such peptides may easily be synthesized or produced by recombinant means to produce a binding agent useful according to the invention. Such methods are well known to those of ordinary skill in the art. Peptides can be synthesized for example, using automated peptide synthesizers which are commercially available. The peptides can be produced by recombinant techniques by incorporating the DNA expressing the peptide into an expression vector and transforming cells with the expression vector to produce the peptide.

Hence, the present invention relates to any binding molecule, antibody or binding fragment obtainable in accordance with above described means and display the mentioned properties.

In a further embodiment of the present invention, the binding molecule, antibody, immunoglobulin chain or a binding fragment thereof or the antigen is detectably labeled. Labeling agents can be coupled either directly or indirectly to the antibodies or antigens of the invention. One example of indirect coupling is by use of a spacer moiety. Furthermore, the antibodies of the present invention can comprise a further domain, said domain being linked by covalent or non-covalent bonds. The linkage can be based on genetic fusion according to the methods known in the art and described above or can be performed by, e.g., chemical cross-linking as described in, e.g., international application WO 94/04686. The additional domain present in the fusion protein comprising the antibody of the invention may preferably be linked by a flexible linker, advantageously a polypeptide linker, wherein said polypeptide linker comprises plural, hydrophilic, peptide-bonded amino acids of a length sufficient to span the distance between the C-terminal end of said further domain and the N-terminal end of the antibody of the invention or vice versa. The therapeutically or diagnostically active agent can be coupled to the antibody of the invention or an antigen-binding fragment thereof by various means. This includes, for example, single-chain fusion proteins comprising the variable regions of the antibody of the invention coupled by covalent methods, such as peptide linkages, to the therapeutically or diagnostically active agent. Further examples include molecules which comprise at least an antigen-binding fragment coupled to additional molecules covalently or non-covalently include those in the following non-limiting illustrative list. Traunecker, Int. J. Cancer Surp. SuDP 7 (1992), 51-52, describe the bispecific reagent janusin in which the Fv region directed to CD3 is coupled to soluble CD4 or to other ligands such as OVCA and IL-7. Similarly, the variable regions of the antibody of the invention can be constructed into Fv molecules and coupled to alternative ligands such as those illustrated in the cited article. Higgins, J. Infect. Dis. 166 (1992), 198-202, described a hetero-conjugate antibody composed of OKT3 cross-linked to an antibody directed to a specific sequence in the V3 region of GP120. Such hetero-conjugate antibodies can also be constructed using at least the variable regions contained in the antibody of the invention methods. Additional examples of specific antibodies include those described by Fanger, Cancer Treat. Res. 68 (1993), 181-194 and by Fanger, Crit. Rev. Immunol. 12 (1992), 101-124. Conjugates that are immunotoxins including conventional antibodies have been widely described in the art. The toxins may be coupled to the antibodies by conventional coupling techniques or immunotoxins containing protein toxin portions can be produced as fusion proteins. The antibodies of the present invention can be used in a corresponding way to obtain such immunotoxins. Illustrative of such immunotoxins are those described by Byers, Seminars Cell. Biol. 2 (1991), 59-70 and by Fanger, Immunol. Today 12 (1991), 51-54.

The above described fusion protein may further comprise a cleavable linker or cleavage site for proteinases. These spacer moieties, in turn, can be either insoluble or soluble (Diener et al., Science 231 (1986), 148) and can be selected to enable drug release from the antibody at the target site. Examples of therapeutic agents which can be coupled to the antibodies of the present invention for immunotherapy are drugs, radioisotopes, lectins, and toxins. The drugs with which can be conjugated to the antibodies and antigens of the present invention include compounds which are classically referred to as drugs such as mitomycin C, daunorubicin, and vinblastine. In using radioisotopically conjugated antibodies or antigens of the invention for, e.g., immunotherapy, certain isotopes may be more preferable than others depending on such factors as leukocyte distribution as well as stability and emission. Depending on the autoimmune response, some emitters may be preferable to others. In general, α and β particle emitting radioisotopes are preferred in immunotherapy. Preferred are short range, high energy a emitters such as ²¹²Bi. Examples of radioisotopes which can be bound to the antibodies or antigens of the invention for therapeutic purposes are ¹²⁵I, ¹³¹I, ⁹⁰I, ⁶⁷Cu, ²¹²Bi, ²¹²At, ²¹¹Pb, ⁴⁷Sc, ¹⁰⁹Pd and ¹⁸⁸Re. Most preferably, the radiolabel is ⁶⁴Cu. Other therapeutic agents which can be coupled to the antibody or antigen of the invention, as well as ex vivo and in vivo therapeutic protocols, are known, or can be easily ascertained, by those of ordinary skill in the art. Wherever appropriate the person skilled in the art may use a polynucleotide of the invention encoding any one of the above described antibodies, antigens or the corresponding vectors instead of the proteinaeous material itself.

The antibody of the present invention can be labeled (e.g., fluorescent, radioactive, enzyme, nuclear magnetic, heavy metal) and used to detect specific targets in vivo or in vitro including “immunochemistry” like assays in vitro.

Due to the provision of the subject antibodies having unique properties and in particular by disclosing the variable region and complementarity determining region (CDRs) of the subject antibody as well as the epitope recognized by the antibody the present invention also provides the necessary structural information for and thus extends to anti-idiotypic antibodies and (synthetic) peptides or peptide-based compounds comprising an epitope specifically recognized by an antibody of the present invention. Anti-idiotypic antibodies and equivalent binding molecules as well as epitopes of the present invention are particularly useful for diagnostic purposes, for example in the detection and isolation of protective anti-TMEFF2 autoantibodies in humans.

The term “peptide” is intended to also refer to the products of post-expression modifications of the TMEFF2 polypeptide, including without limitation glycosylation, acetylation, phosphorylation, amidation, derivatization by known protecting/blocking groups, proteolytic cleavage, or modification by non-naturally occurring amino acids. A peptide may be derived from a natural biological source or produced by recombinant technology, but is not necessarily translated from a designated nucleic acid sequence. It may be generated in any manner, including by chemical synthesis. The peptide of the invention may be of a size of about 5 or more, 7 or more, 10 or more, 20 or more, 25 or more, 50 or more, 75 or more, 100 or more but preferably less than 95, more preferably less than 50 and most preferably less than 25 amino acids.

From the foregoing, it is evident that the present invention encompasses any use of an TMEFF2 binding molecule comprising at least one CDR of the above described antibody, in particular for diagnosing and/or treatment of an affective and/or anxiety disorder as mentioned above. Preferably, said binding molecule is an antibody of the present invention or an immunoglobulin chain thereof. In addition, the present invention relates to anti-idiotypic antibodies of any one of the mentioned antibodies described above. These are antibodies or other binding molecules which bind to the unique antigenic peptide sequence located on an antibody's variable region near the antigen-binding site and are useful, e.g., for the detection of anti-TMEFF2 antibodies in sample of a subject.

In another embodiment the present invention relates to a diagnostic composition comprising any one of the above described TMEFF2 binding molecules, antibodies, antigen-binding fragments, polynucleotides, vectors or cells of the invention and optionally suitable means for detection such as reagents conventionally used in immuno or nucleic acid based diagnostic methods. The antibodies of the invention are, for example, suited for use in immunoassays in which they can be utilized in liquid phase or bound to a solid phase carrier. Examples of immunoassays which can utilize the antibody of the invention are competitive and non-competitive immunoassays in either a direct or indirect format. Examples of such immunoassays are the radioimmunoassay (RIA), the sandwich (immunometric assay), flow cytometry and the Western blot assay. The antigens and antibodies of the invention can be bound to many different carriers and used to isolate cells specifically bound thereto. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention. There are many different labels and methods of labeling known to those of ordinary skill in the art. Examples of the types of labels which can be used in the present invention include enzymes, radioisotopes, colloidal metals, fluorescent compounds, chemiluminescent compounds, and bioluminescent compounds; see also the embodiments discussed hereinabove.

By a further embodiment, the TMEFF2 binding molecules, in particular antibodies of the present invention may also be used in a method for the diagnosis of a disorder in an individual by obtaining a body fluid sample from the tested individual which may be a blood sample, a lymph sample or any other body fluid sample and contacting the body fluid sample with an antibody of the instant invention under conditions enabling the formation of antibody-antigen complexes. The level of such complexes is then determined by methods known in the art, a level significantly higher than that formed in a control sample indicating the disease in the tested individual. In the same manner, the specific antigen bound by the antibodies of the invention may also be used. Thus, the present invention relates to an in vitro immunoassay comprising the binding molecule, e.g., antibody or antigen-binding fragment thereof of the invention.

As used herein, the term “sample” refers to any biological material obtained from a subject or patient, cell line, tissue culture, or other source containing polynucleotides or polypeptides or portions thereof. In one aspect, a sample can comprise blood, cerebrospinal fluid (“CSF”), sera, plasma, urine, synovial fluid, spinal fluid or urine. In other aspects, a sample can comprise whole blood, plasma, B cells enriched from blood samples, and cultured cells (e.g., B cells from a subject). A sample can also include a biopsy or tissue sample including neural tissue. Methods for obtaining tissue biopsies and body fluids from mammals are well known in the art. In still other aspects, a sample can comprise whole cells and/or a lysate of the cells. Blood samples can be collected by methods known in the art. In one aspect, the pellet can be resuspended by vortexing at 4° C. in 200 μl buffer (20 mM Tris, pH. 7.5, 0.5% Nonidet, 1 mM EDTA, 1 mM PMSF, 0.1 M NaCl, IX Sigma Protease Inhibitor, and IX Sigma Phosphatase Inhibitors 1 and 2). The suspension can be kept on ice for 20 minutes with intermittent vortexing. After spinning at 15,000×g for 5 minutes at about 4° C., aliquots of supernatant can be stored at about −70° C. In a preferred embodiment of the described method of diagnosis the individual is a mammal and more preferably human. Moreover, the cells are preferably derived from skin, blood, urine or cerebral spinal fluid or the pituitary glands.

In this context, the present invention also relates to means specifically designed for this purpose. For example, an antibody-based array may be used, which is for example loaded with antibodies or equivalent antigen-binding molecules of the present invention which specifically recognize TMEFF2. Design of microarray immunoassays is summarized in Kusnezow et al., Mol. Cell Proteomics 5 (2006), 1681-1696. Accordingly, the present invention also relates to microarrays loaded with TMEFF2 binding molecules identified in accordance with the present invention.

Thus, the level of TMEFF2 may be assessed by any suitable method known in the art comprising, e.g., analyzing TMEFF2 by one or more techniques chosen from Western blot, immunoprecipitation, enzyme-linked immunosorbent assay (ELISA), radioimmunoassay (RIA), fluorescent activated cell sorting (FACS), two-dimensional gel electrophoresis, mass spectroscopy (MS), matrix-assisted laser desorption/ionization-time of flight-MS (MALDI-TOF), surface-enhanced laser desorption ionization-time of flight (SELDI-TOF), high performance liquid chromatography (HPLC), fast protein liquid chromatography (FPLC), multidimensional liquid chromatography (LC) followed by tandem mass spectrometry (MS/MS), and laser densitometry. Preferably, said in vivo imaging of TMEFF2 comprises positron emission tomography (PET), single photon emission tomography (SPECT), near infrared (NIR) optical imaging or magnetic resonance imaging (MRI).

Methods of diagnosing affective and anxiety disorder as defined above for monitoring a TMEFF2-related disease progression, and monitoring a TMEFF2 related disease treatment using antibodies and related means which may be adapted in accordance with the present invention are also described in international application WO 2007/090631, the disclosure content of which is incorporated herein by reference. These methods may be applied as described but with a TMEFF2 specific antibody, binding fragment, derivative or variant of the present invention.

The present invention also provides a pharmaceutical and diagnostic, respectively, pack or kit comprising one or more containers filled with one or more of the above described ingredients, i.e. binding molecule, antibody or binding fragment thereof, polynucleotide, vector or cell of the present invention. Associated with such container(s) can be a notice in the form prescribed by a governmental agency regulating the manufacture, use or sale of pharmaceuticals or biological products, which notice reflects approval by the agency of manufacture, use or sale for human administration. In addition or alternatively the kit comprises reagents and/or instructions for use in appropriate diagnostic assays. The composition, i.e. kit of the present invention is of course particularly suitable for the diagnosis, prevention and treatment of an affective and/or anxiety disorder which is accompanied with the presence of TMEFF2; see also supra, and in particular applicable for the treatment of major depression, anxiety, dysthymia, atypical depression, premenstrual dysphoric disorder, seasonal affective disorder, and bipolar disorder, depressive episode, recurrent depressive disorder, manic episode, bipolar affective disorder, persistent mood disorders, other mood disorders, unspecified mood disorder, phobic anxiety disorders, other anxiety disorders, obsessive-compulsive disorder, reaction to severe stress and adjustment disorders, dissociative disorders, somatoform disorders, other neurotic disorders

It has been discovered in accordance with the present invention that effective treatment regimens for TMEFF2 antagonist, in particular anti-TMEFF2 antibody in the treatment of affective and/or anxiety disorder do exist. This has been exemplified with a murine anti-human TMEFF2 antibody in mouse models; see Examples 4 to 6. Accordingly, it is reasonable to expect that the results obtained for the murine anti-human TMEFF2 antibody in mice are transferable to the human anti-human TMEFF2 antibody as well as to humanized and fully human versions thereof in the treatment of affective and/or anxiety disorders in humans.

The terms “treatment”, “treating”, and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease.

By “subject” or “individual” or “animal” or “patient” or “mammal,” is meant any subject, particularly a mammalian subject, e.g., a human patient, for whom diagnosis, prognosis, prevention, or therapy is desired.

The pharmaceutical compositions of the present invention can be formulated according to methods well known in the art; see for example Remington: The Science and Practice of Pharmacy (2000) by the University of Sciences in Philadelphia, ISBN 0-683-306472. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intra-muscular, topical or intradermal administration. Aerosol formulations such as nasal spray formulations include purified aqueous or other solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier.

Furthermore, whereas the present invention includes the now standard (though fortunately infrequent) procedure of drilling a small hole in the skull to administer a drug of the present invention, in a preferred aspect, the binding molecule, especially antibody or antibody based drug of the present invention can cross the blood-brain barrier, which allows for intravenous or oral administration.

In one embodiment of the present invention the pharmaceutical or diagnostic composition may be formulated comprising anti-TMEFF2 antibody or binding fragment, antigen-binding molecule derivative or variant thereof a pharmaceutically acceptable carrier for nasal administration or injection, preferably for extended release.

In a preferred embodiment the antibody is formulated for intranasal delivery. For this purpose, the antibody may be present in a solution suitable for intranasal administration such as saline. Formulations for intranasal administration may also contain, in addition to the antibody, one or more ingredients selected from the group consisting of bile salts, alkyl glycosides, polymers, gelatin and/or chitosan, tight junction modulating peptides, lipids and surfactants, cyclodextrins and chelators.

In a further embodiment, a pharmaceutical composition for intranasal administration contains a penetration enhancer like Pz-peptide (4-Phenylazobenzoxycarbonyl-Pro-Leu-Gly-Pro-D-Arg; Bachem, Bubendorf, Switzerland) and/or mucoadhesives like sodium hyaluronate, chitosan, lectins. It is also conceivable that the antibody is encapsulated or coupled in liposome carriers, microspheres, or particulate vectors such as microemulsions or nanoemulsions and nanoparticles.

Formulations suitable for intranasal administration may also make use of nanoparticle systems, including nanoparticles with surface modifications by chitosan, PEG, lectin; poly/oligosaccharide nanoparticles composed of chitosan and cyclodextrins; nanoparticles coated with ligands like ulex europeus agglutinin I (UEA I) or wheat germ agglutinin-horseradish peroxidase (WGA), or UEA I or WGA conjugated PEG-PLA (polylactic acid nanoparticles coated with a hydrophilic polyethyleneglycol) nanoparticles, or alternatively nanoparticles coated with olfactory receptor neuron (ORN) “homing peptides”, such as the phage-selected ACTTPHAWLCG peptide (SEQ ID NO: 19).

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered falls within the ranges indicated. Progress can be monitored by periodic assessment. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Furthermore, the pharmaceutical composition of the invention may comprise further agents which are suitable to treat an affective and/or anxiety disorders and the intended use of the pharmaceutical composition. For example, for use in the treatment of affective and/or anxiety disorders the additional agent may be selected from the group consisting of small molecules, anti-TMEFF2 antibodies, and combinations thereof. Preferably, such a molecule or compound is selected from the group consisting of amitriptyline, amitriptyline oxide, desipramine, dibenzepin, dosulepin, doxepin, chloroimipramine, imipramine, nortriptyline, mianserin, maprotiline, trimipramine, CP-122721, elzasonan, PD-171729, MK-869, DOV-216303, DOV-21947, licarbazepine, amfebutamone, radafaxine, vilazodone, GSK-679769, GW-597599, NS-2359, GSK-876008, pramipexole, duloxetine, atomoxetine, LY-628535, desvenlafaxine, escitalopram, LU-AA21004, saredutant, SR-58611, SSR-149415, SSR-146977, moclobemide, R-673, R-1204, BMS-469458, DPC-368, Org-34517, Org-34850, inhibitors of the CRH receptors, ONO-2333Ms, NBI-876008, AAG-561, NBI-34041, DPC-368, PD-171729, SSR-125543, viloxazine, trazodone, nefazodone, mirtazapine, venlafaxine, reboxetine, tranylcypromine, brofaromine, moclobemide, citalopram, paroxetine, fluoxetine, fluvoxamine, sertraline, Hypericum (St. John's Wort), alprazolam, clonazepam, diazepam, lorazepam, halazepam, chlordiazepoxide, and other drugs such as buspirone, clonidine, pagoclone, risperidone, olanzapine, quetiapine, ziprasidone, celecoxib, piroxicam, parecoxib, valdecoxib, PMI-001, PH-686464, SC-58236, etoricoxib, rofecoxib, L-776967, lumiracoxib, GW-406381, GW-644784, meloxicam, SVT-2016, PAC-10649, CS-706, LAS-34475, cimicoxib, A-183827.0, or nimesulide. Furthermore, it is envisaged that the anti-TMEFF2 antibody and like molecule of the present invention and a further compound suitable for treating an affective and/or anxiety disorder are administered simultaneously, sequentially or separately from each other.

In one embodiment, it may be beneficial to use recombinant Fab (rFab) and single chain fragments (scFvs) of the antibody of the present invention, which might more readily penetrate a cell membrane. The perceived advantages of using small Fab and scFv engineered antibody formats which lack the effector function include more efficient passage across the blood-brain barrier and minimizing the risk of triggering inflammatory side reactions. Furthermore, besides scFv and single-domain antibodies retain the binding specificity of full-length antibodies, they can be expressed as single genes and intracellularly in mammalian cells as intrabodies, with the potential for alteration of the folding, interactions, modifications, or subcellular localization of their targets; see for review, e.g., Miller and Messer, Molecular Therapy 12 (2005), 394-401.

In a different approach Muller et al., Expert Opin. Biol. Ther. (2005), 237-241, describe a technology platform, so-called ‘SuperAntibody Technology’, which is said to enable antibodies to be shuttled into living cells without harming them. Such cell-penetrating antibodies open new diagnostic and therapeutic windows. The term ‘TransMabs’ has been coined for these antibodies.

In addition, co-administration or sequential administration of other agents may be desirable.

Hence, in a particular preferred embodiment the present invention relates to the use of the TMEFF2 binding molecule, e.g., antibody or antigen-binding fragment thereof of the present invention or of a binding molecule having substantially the same binding specificities of any one thereof, the polynucleotide, the vector, the cell or the anti-idiotypic antibody or the peptide or peptide-based compound of the present invention for the preparation of a pharmaceutical or diagnostic composition for prophylactic and therapeutic treatment of an affective and/or anxiety disorder, monitoring the progression of a TMEFF2-related disorder a response to a TMEFF2-related disease in a subject or for determining a subject's risk for developing an affective and/or anxiety disorder.

Hence, in one embodiment the present invention relates to a method of treating affective and/or anxiety disorders characterized by comprising major depression, anxiety, dysthymia, atypical depression, premenstrual dysphoric disorder, seasonal affective disorder, and bipolar disorder, depressive episode, recurrent depressive disorder, manic episode, bipolar affective disorder, persistent mood disorders, other mood disorders, unspecified mood disorder, phobic anxiety disorders, other anxiety disorders, obsessive-compulsive disorder, reaction to severe stress and adjustment disorders, dissociative disorders, somatoform disorders, other neurotic disorders.

As described above, the antibody molecules according to the present invention are effective TMEFF2 antagonists in the sense that they interfere with the binding of TMEFF2 to Activin thereby leading to an increase in SMAD signaling which in turn indicates antidepressant and anxiolytic properties while the locomotor behavior of a subject remains unaffected. The method comprises administering to a subject in need thereof a therapeutically effective amount of any one of the afore-described TMEFF2 binding molecules, antibodies, polynucleotides, vectors or cells of the instant invention.

A therapeutically effective dose or amount refers to that amount of the active ingredient sufficient to ameliorate the symptoms or condition. Therapeutic efficacy and toxicity of such compounds can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, LD₅₀/ED₅₀. Preferably, the therapeutic agent in the composition is present in an amount sufficient to restore or preserve normal behavior and/or cognitive properties in case of affective and/or anxiety disorders or other TMEFF2-related disorders.

These and other embodiments are disclosed and encompassed by the description and Examples of the present invention. Further literature concerning any one of the materials, methods, uses and compounds to be employed in accordance with the present invention may be retrieved from public libraries and databases, using for example electronic devices. For example the public database “Medline” may be utilized, which is hosted by the National Center for Biotechnology Information and/or the National Library of Medicine at the National Institutes of Health. Further databases and web addresses, such as those of the European Bioinformatics Institute (EBI), which is part of the European Molecular Biology Laboratory (EMBL) are known to the person skilled in the art and can also be obtained using internet search engines. An overview of patent information in biotechnology and a survey of relevant sources of patent information useful for retrospective searching and for current awareness is given in Berks, TIBTECH 12 (1994), 352-364.

The above disclosure generally describes the present invention. Unless otherwise stated, a term as used herein is given the definition as provided in the Oxford Dictionary of Biochemistry and Molecular Biology, Oxford University Press, 1997, revised 2000 and reprinted 2003, ISBN 0 19 850673 2. Several documents are cited throughout the text of this specification. Full bibliographic citations may be found at the end of the specification immediately preceding the claims. The contents of all cited references (including literature references, issued patents, published patent applications as cited throughout this application and manufacturer's specifications, instructions, etc) are hereby expressly incorporated by reference; however, there is no admission that any document cited is indeed prior art as to the present invention.

A more complete understanding can be obtained by reference to the following specific Examples which are provided herein for purposes of illustration only and are not intended to limit the scope of the invention.

### EXAMPLES

The examples which follow further illustrate the invention, but should not be construed to limit the scope of the invention in any way. Detailed descriptions of conventional methods, such as those employed herein can be found in the cited literature; see also “The Merck Manual of Diagnosis and Therapy” 17^{th} Ed. edited by Beers and Berkow (Merck & Co., Inc. 2003).

The practice of the present invention will employ, unless otherwise indicated, conventional techniques of cell biology, cell culture, molecular biology, transgenic biology, microbiology, recombinant DNA, and immunology, which are within the skill of the art. For further elaboration of general techniques useful in the practice of this invention, the practitioner can refer to standard textbooks and reviews in cell biology and tissue culture; see also the references cited in the examples. General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); DNA Cloning, Volumes I and II (Glover ed., 1985); Oligonucleotide Synthesis (Gait ed., 1984); Nucleic Acid Hybridization (Hames and Higgins eds. 1984); Transcription And Translation (Hames and Higgins eds. 1984); Culture Of Animal Cells (Freshney and Alan, Liss, Inc., 1987); Gene Transfer Vectors for Mammalian Cells (Miller and Calos, eds.); Current Protocols in Molecular Biology and Short Protocols in Molecular Biology, 3rd Edition (Ausubel et al., eds.); and Recombinant DNA Methodology (Wu, ed., Academic Press). Gene Transfer Vectors For Mammalian Cells (Miller and Calos, eds., 1987, Cold Spring Harbor Laboratory); Methods In Enzymology, Vols. 154 and 155 (Wu et al., eds.); Immobilized Cells And Enzymes (IRL Press, 1986); Perbal, A Practical Guide To Molecular Cloning (1984); the treatise, Methods In Enzymology (Academic Press, Inc., N.Y.); Immunochemical Methods In Cell And Molecular Biology (Mayer and Walker, eds., Academic Press, London, 1987); Handbook Of Experimental Immunology, Volumes I-IV (Weir and Blackwell, eds., 1986). Protein Methods (Bollag et al., John Wiley & Sons 1996); Non-viral Vectors for Gene Therapy (Wagner et al. eds., Academic Press 1999); Viral Vectors (Kaplitt & Loewy eds., Academic Press 1995); Immunology Methods Manual (Lefkovits ed., Academic Press 1997); and Cell and Tissue Culture: Laboratory Procedures in Biotechnology (Doyle & Griffiths, John Wiley & Sons 1998). Reagents, cloning vectors and kits for genetic manipulation referred to in this disclosure are available from commercial vendors such as BioRad, Stratagene, Invitrogen, Sigma-Aldrich, and ClonTech. General techniques in cell culture and media collection are outlined in Large Scale Mammalian Cell Culture (Hu et al., Curr. Opin. Biotechnol. 8 (1997), 148); Serum-free Media (Kitano, Biotechnology 17 (1991), 73); Large Scale Mammalian Cell Culture (Curr. Opin. Biotechnol. 2 (1991), 375); and Suspension Culture of Mammalian Cells (Birch et al., Bioprocess Technol. 19 (1990), 251); Extracting information from cDNA arrays, Herzel et al., CHAOS 11 (2001), 98-107.

The Examples which follow further illustrate the invention, but should not be construed to limit the scope of the invention in any way. The following experiments are illustrated and described with respect to antibody PQ01.

### Example 1

### Generation of Antibodies Against TMEFF2

Monoclonal antibodies against TMEFF2 were generated by applying the hybridoma technology. To this end, three Balb/c mice were immunized with a recombinant protein corresponding to amino acids 166-262 of the TMEFF2 protein amino acid sequence depicted in SEQ ID NO: 1 (sequence of recombinant protein: Q F G A E C D E D A E D V W C V C NIDCSQTNFNPLCASDGKSYDNACQIKEASCQKQEKIEVMSL GRCQDNTTTTTKSEDGHYARTDYAENANKLEESAREHH; SEQ ID NO: 18). Lymph nodes cells from these immunized mice are isolated and then fused with the myeloma cell line P3-X63-Ag8 according to standard procedures. The resulting supernatants of mixed hybridoma clones were screened by ELISA and immunofluorescence on NIH 3T3 cells overexpressing the full length TMEFF2 protein in order to identify and select anti-TMEFF2 antibody-producing clones. Selected positive clones were then twice subcloned to monoclonality and their properties were further assessed. The resulting anti-TMEFF2 monoclonal antibodies were subsequently characterized by western blot analysis.

Briefly, CHO-K1 cells overexpressing TMEFF2 were harvested, centrifuged, resuspended, and homogenized in a Protease Inhibitor Mix Solution according to the manufacturer specifications (Sigma Aldrich). Resulting protein homogenates were then separated by sodium dodecyl sulfate polyacrylamide gel electrophoresis (4-12% Bis-Tris SDS-PAGE gradient; 100 μg protein homogenate per well) for 1 hour at 200 Volts. Separated proteins were then transferred onto a Hybond ECL nitrocellulose membrane (Amersham) by electroblotting using the semi-wet transfer unit XCellII Blot Module (InvitroGen) for 1 hour at 24 Volts. The nitrocellulose membrane was then washed 1 time for 5 minutes in Tris Buffered Saline (TBS) buffer. Blocking of non-specific binding was achieved by placing the membrane in 5% non-fat dry milk prepared TBS for 1 hour. The membrane was then incubated with the TMEFF2 monoclonal antibody (1:1000 dilution in 5% non fat dry milk) overnight at room temperature. The membrane was then washed 5 times for 5 minutes in TBS before being incubated with an ECL anti-mouse IgG, horse radish peroxidase linked secondary antibody (1:2000 dilution in 5% non fat dry milk; Amersham) for 1 hour at room temperature. The membrane was then washed 5 times for 5 minutes in TBS and then incubated for 5 minutes in Lumi-Light Chemiluminescence POD substrate (Roche Applied Science) and then exposed on a Lumi-film Chemiluminescent detection film (Roche Applied Science). As demonstrated in FIG. 2 for one sample designated PQ01 the antibody recognized a 43 kDa band in TMEFF2 overexpressing cells corresponding to the size of the TMEFF2 protein.

### Example 2

### Detection of and Isolation of High-Affinity Anti-TMEFF2-Specific Antibodies by ELISA

An enzyme-linked immunosorbent assay (ELISA) is established to measure the binding of different anti-TMEFF2 antibodies to their respective antigen. It is based on the ELISA described in Ternant et al., Ther. Drug Monit. 28 (2006), 169-174. In particular, 96-well plates (Maxisorp, Nunc, #735-0083) were coated with antigen (either a 97aa peptide, representing aa 166-262 of TMEFF2, or the full-length TMEFF2 protein, Abnova #H00023671-P01; 1 μg/m in PBS, 50 μl per well) for 1.5 hour at 37° C. or overnight at 4° C. Thereafter, the plates are washed 3 times with 300 μl washing buffer (PBS+0.05% Tween 20). After washing, the plates are blocked with 300 μl blocking buffer (PBS+5% milk powder) per well for 30 minutes at room temperature, followed by a washing step with 3×300 μl washing buffer. Antibodies are diluted in reagent buffer (PBS+0.5% milk powder) to the respective concentration (FIG. 3: 500 ng/ml of different antibodies obtained in the screening procedure described in Example 1; FIG. 4: 2 ng/ml-500 ng/ml of different antibodies obtained in the screening procedure described in Example 1, FIG. 5: 500 ng/ml of antibody PQ01 or commercially available anti-TMEFF2). The 1D12 antibody IgG2a Kappa of Abnova was raised against the Immunogen TMEFF2 (NP_{—}057276.2, 201 aa˜292 aa having the Sequence SYDNACQIKEASCQKQEKIEVMSLGRCQDNTTTTTKSEDGHYARTDYAENANKLEES AREHHIPCPEHYNGFCMHGKCEHSINMQEPSCRCD (SEQ ID NO: 20). Then, the samples are applied to the plate (50 μl per well) and incubated for 1 hour at room temperature. After washing with 3 times with 300 μl washing buffer, the secondary antibody goat anti-mouse IgG-POD-conjugated (Jackson Immuno Research #115-035-205) is applied at a dilution of 1:50000 in blocking buffer (50 μl per well) and the plates are incubated 1 hour at room temperature. The plates are then washed again with 3 times with 300 μl washing buffer and 50 μl per well of the chromogenic substrate TMB (Sigma #T444) are added subsequently. The plates are incubated 5-10 minutes at 37° C. and the color reaction is stopped by adding 200 μl of 2 M sulfuric acid (H₂SO₄, Sigma-Aldrich #258105) to every well. Finally, the plates are measured with an ELISA Reader (FLUOstar OPTIMA, BMG Labtech) at 450 nm wavelength.

The signals obtained with various TMEFF2 antibodies produced by the hybridoma technology as described in Example 1 are not only concentration-dependent but also reflect sub-clone specific binding properties (FIG. 3, 4), with the antibody designated PQ01 (#001) showing binding already at a concentration of 2 ng/ml. Further, using the full length TMEFF2 protein (Abnova) as antigen, the ELISA signal for PQ01 is considerably higher than that of commercially available anti-TMEFF2 antibodies (FIG. 5).

Because of its properties the antibody PQ01 was chosen for further investigations. The nucleotide sequence coding for the antibody PQ01 was cloned and the nucleotide sequence encoding the variable regions of the light and heavy chain were determined according to standard methods. The nucleotide and amino acid sequences of the variable regions of the heavy and light chain of PQ01 are shown in FIG. 1 and the complementarity determining regions (CDRs) (according to Kabat) are underlined.

### Example 3

### PQ01 Recognizes a Linear Epitope Comprising a Core Sequence of 7 Amino Acids

The identification of epitopes or immunodominant regions in antigens represents an important step in characterization of antibodies. A very efficient way to identify such epitopes is incubation of a collection of antigen derived peptides displayed on peptide microarrays with antibodies of interest.

The determination of peptide-antibody binding was performed by RepliTope-analysis where the peptide microarray was incubated with the target antibody followed by a fluorescently labeled secondary antibody directed against the Fc-part of the primary one. The specific signals are measured by means of a high resolution microarray scanning system.

For this RepliTope experiment the following sequence of the protein TMEFF2_Human QFGAECDEDAEDVWC VCNIDCSQTNFNPLC ASDGKSYDNACQIKE ASCQKQEKIEVMSLG RCQDNTTTTTKSEDG HYARTDYAENANKLE ESAREHH (SEQ ID NO: 18) was scanned in format 15/11 resulting in a total of 22 peptides.

All peptides are synthesized in a stepwise manner on a cellulose membrane. By coupling a reactivity tag (tag+linker) on the N-terminus of the peptides (truncated side products are capped by acetylation steps), all target-peptides are immobilized chemoselectively and purified by reaction of the peptides with the modified glass surface. The resulting formation of a covalent bond between the target peptide and the chip surface allows removal of all truncated (and acetylated) sequences by subsequent washing steps. The peptide microarray can be incubated with blocking buffer (Candor Biosciences, SmartBlock, #113 125) for two hours to reduce non-specific binding of the antibody. Subsequently, the peptide microarray chips are incubated with individual target antibodies diluted in diluent buffer (Pierce International, Superblock TBS, #37536; 1 μg/mL, total assay volume 200 μL) or with diluent only (control), using a TECAN HS4800 mircorarray processing station. Subsequent to incubation with target antibodies the microarrays are washed three times with TBS-buffer including 0.1% Tween20 (JPT) followed by an incubation with fluorescently-labelled secondary antibody (Anti-mouse-IgG, Thermo Scientific 35515, labeled with Dylight647; 1 μg/mL diluent buffer). Microarrays are washed 3 times with TBS-buffer and SSC-buffer (3 mM, JPT) and dried using a nitrogen stream.

After performing the incubation steps and subsequent to the final washing steps the microarrays are dried and scanned in a high resolution microarray scanning system (Axon GenePix Scanner 4200AL). The resulting image is processed and analyzed using spot-recognition software GenePix 7, showing the signal intensity (Light Units, LU) as single measurements for each peptide. Each spot-feature was analyzed for total intensity and background intensity. All data are corrected for local background of each feature, according to the algorithm applied in the spot recognition software. For data analysis the median of signal intensities for pixels around recognized spots (background) was subtracted from median of signal intensities for pixels within recognized spots (signal) resulting in corrected median values (signal minus background). Mean values of corrected median of signal intensities from 3 identical subarrays and from 3 spots per subarray on each microarray image are used for data evaluation (mean of 9 datapoints per microarray).

The results of this epitope mapping experiment using the antibody PQ01 are shown in FIG. 6.

The control incubation showed no signals on the peptide library. Incubation with target antibody PQ01 yields significant signals on peptides 17, 18, 19 and on peptides 2 and 3, the latter with very weak intensity.

| (SEQ ID NO: 14) | |
|---|---|
| Peptide 17 | NTTTTTKSEDGHYAR |
| (SEQ ID NO: 15) | |
| Peptide 18 | TTKSEDGHYARTDYA |
| (SEQ ID NO: 16) | |
| Peptide 19 | EDGHYARTDYAENAN |

Thus, antibody PQ01 shows significant binding to an epitope contained in peptides 17, 18, and 19 as also depicted in FIG. 6. The overlapping sequence in these peptides is EDGHYAR (SEQ ID NO: 13) and the overall sequence represented by peptides 17, 18, and 19 is NTTT TTKS EDGH YART DYAE NAN (SEQ ID NO: 17).

### Example 4

### PQ01 Reduces Passive and Increases Active Escape-Oriented Behaviour in the Forced Swim Test (FST)

The effects of the monoclonal TMEFF2 antibody PQ01 on depressive-like behavior is assessed using the forced swim test paradigm. The forced swim test is a standard test that is based on the assumption that animals will normally try to escape from an aversive situation. When the aversive stimulation is inescapable, the animal will eventually stop trying to escape. Early cessation of attempts to escape is considered a rodent analogue of stress-induced depression. The test is used to determine the effectiveness of antidepressants, test new pharmaceutical compounds and validate animal models of depression (Porsolt et al., Arch. Int. Pharmacodym. 229 (1977), 327-336; Porsolt, Rev. Neurosci. 11 (2000), 53-58; Rénéric et al., Behav. Brain Res. 136 (2002), 521-532; Page et al., Psychopharmacology 165 (2003), 194-201; Kelliher et al., Psychoneuroendocrinology 28 (2003), 332-347). The test consists of placing a mouse for a period of 5 minutes into a glass cylinder containing water. Under such circumstances, the mouse cannot touch the bottom of the cylinder and is thus forced to swim. Time, latency and frequency of struggling versus floating are scored as behavioral parameters. Floating (i.e. movements made only for keeping balance and breath) is a passive behavior associated with despair and represents a depressive-like symptom since the animal does not make any effort to actively cope with the stressful situation. Increased struggling (i.e. active attempts to escape) indicates active coping behavior that can be interpreted as an improvement of depression-like symptoms. The forced swim test is sensitive to all major classes of antidepressants, including tricyclics, selective norepinephrine and serotonin reuptake inhibitors, monoamine oxidase inhibitors and atypical antidepressants (Lucki et al., Psychopharmacology 155 (2001), 315-322). Different mouse strains vary in their responsiveness to antidepressants. A very well suited strain to detect antidepressant drug properties are DBA/2 mice, as it has been shown that they respond to treatment with antidepressants with various modes of action like e.g. the selective serotonin and norepinephrine reuptake inhibitors Fluoxetine and Desipramine (Lucki et al., Psychopharmacology 155 (2001), 315-322) and the mood stabilizer Lithium (Can et al., Genes Brain. Behay. 10 (2011), 434-443).

Briefly, female DBA/2 mice are divided into two groups of 10 mice each. Individuals of one group are treated with TMEFF2 antibody PQ01 while the other group is treated with vehicle (PBS-buffer). Each mouse is injected three times: The first injection is given intravenously, while two and six days later the injection is applied intraperitoneal.

The dosage can be 100 μg per injection using PBS (1×, Dulbecco's PBS, sterile, from PAA Laboratories, # H15-002) as a vehicle and at a concentration of 1 μg/ul antibody. The injection volume can be 100 μl.

The latter two injections are followed by a 30 minutes restraint stress. 24 and 48 hours after the third injection forced swim tests are performed. As displayed in FIG. 7, application of the TMEFF2 antibody PQ01 significantly increased active escape behavior (i.e. time struggling), while passive stress coping behavior (i.e. time floating) is decreased when compared to the vehicle control group. These results demonstrate antidepressant properties of the TMEFF2 antibody PQ01 resulting in improvements of depression-like behavior in a well-established rodent model.

### Example 5

### The Antibody PQ01 does not Change Locomotor Activity in Behavioral Experiments (Open Field Test)

After the forced swim test (see above), in the same mice the effects of the TMEFF2 antibody PQ01 on locomotor activity was assessed in a standard test paradigm, the open field. Briefly, 24 hours after the second forced swim test (i.e. 72 hours after the third injection), the mice were individually placed in an open topped, grey lacquered, wooden box (30×30×40 cm, illuminated with 30 lux) and for a period of 30 minutes their locomotor activity was recorded by a video camera mounted above the box. The path length covered was automatically analyzed by a video tracking software.

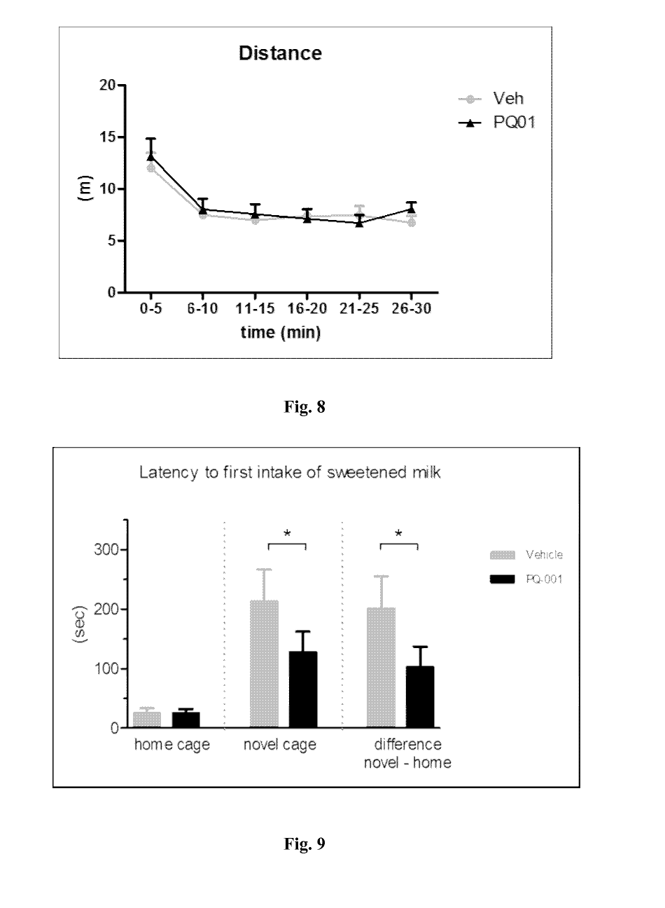

As displayed in FIG. 8, application of the TMEFF2 antibody PQ01 is devoid of any influence on locomotor activity. Further, this demonstrates that i) the increased active escape behavior of PQ01 treated mice observed in the forced swim test reflects true antidepressant effects rather than general changes in activity; and ii) PQ01 does not influence general well-being of the animals.

### Example 6

### PQ01 Reduces Anxiety-Related Behavior in the Novelty-Induced Hypophagia (NIH) Test

The influence of the TMEFF2 antibody PQ01 on anxiety-related behavior can be examined using the novelty-induced hypophagia (NIH) paradigm as described by Dulawa and co-workers (Dulawa et al., Neuropsychopharmacology 29 (2004), 1321-1330; Dulawa and Hen, Neurosci. Biobehav. Rev. 29 (2005), 771-783). Hypophagia refers to inhibition of feeding and is associated with anxiety: more anxious individuals would display a decreased intake of food and palatable drinking fluids in stressful situations. In the NIH paradigm stress is evoked by placing the mice in a novel environment. Before the experiment mice are accustomed to sweetened condensed milk solution, a drinking fluid they are highly motivated to consume. The latency to drink the solution in the home cage is measured. At the testing day each mouse is placed in a novel environment, i.e. an empty cage without bedding, and again the condensed milk solution is presented and the latency to the first consumption is measured. By subtracting the latency measured in the home cage from the latency determined in the novel cage, potential effects of independent factors like differences in appetite or activity are corrected for each individual animal. The resulting latency is a parameter reflecting anxiety with more anxious individuals displaying a longer latency. Vice versa, the latency is decreased after application of anxiolytic drugs or chronic antidepressant drug treatment (Dulawa et al., Neuropsychopharmacology 29 (2004), 1321-1330; Dulawa and Hen, Neurosci. Biobehav. Rev. 29 (2005), 771-783).

Briefly, female DBA/2JIco mice are divided into two groups: Individuals of one group are treated with TMEFF2 antibody (n=14) while the other group is treated with vehicle (PBS-buffer, n=13). Each mouse is treated three times: The first injection was given intravenously, while two and seven days later the injection can be given intraperitoneal.

The dosage can be 100 μg per injection using PBS (lx, Dulbecco's PBS, sterile, from PAA Laboratories, Cat-No. H15-002) as a vehicle and at a concentration of 1 μg/ul antibody. The injection volume can be 100 μl.

The latter two injections are followed by a 30 minutes restraint stress. The mice are habituated to the sweetened condensed milk solution one day before and one day after the last injection. Latency to the consumption of the fluid in the home cage is examined two days after and latency in the novel cage three days after the last injection. The results are illustrated in FIG. 9. The latency to consume the palatable fluid in the novel cage is increased in both groups, showing that this situation was anxiety-provoking. This effect is counteracted by TMEFF2 antibody application, suggesting that treatment with TMEFF2 antibody has anxiolytic properties which are comparable with results of chronic antidepressant treatment.

### Example 7

### PQ01 Increases Activin-Induced Smad-Regulated Signal Pathway Activity in SMAD—Dual-Luciferase Reporter Assay

It had previously been shown that TMEFF2 interacts with Activin which is a member of the transforming growth factor beta (TGF-beta) superfamily. Activin exerts its biological effects by signaling through its types I and II serine/threonine kinase receptor complex and intracellular Smad proteins (Miyazawa et al., Genes Cells 7 (2002), 1191-1204). In order to test the effects of anti-TMEFF2 antibodies on Activin signaling, a SMAD—Dual luciferase Reporter assay in CHO cells overexpressing TMEFF2 can be used. Briefly, for generation of the overexpressing cell line, the TMEFF2 construct was cloned in a Gateway® pcDNA™-DEST40 Vector (Invitrogen, Germany). The Chinese Hamster Ovary cell line derivative K1 (CHO-K1) (ATCC, Manassas, Va.) are cultivated in Ham's F-12 Medium (PAA, Austria) containing 10% Fetal Bovine Serum (PAA) and 2 mM L-Glutamine (PAA), and stably transfected by using the method of Lipofection. The generated clones are maintained in growth medium, supplemented with 500 μg/m G418 (PAA). Clones are characterized by PCR. For performing the assay, the cells are plated in a white 96-well Plate (NUNC, Denmark), 15K per well in Ham's F-12 Medium (PAA) containing 10% Fetal Bovine Serum (PAA) and 2 mM L-Glutamine. At approximately 80% confluence cells are transiently transfected with 100 ng Cignal™Reporter (Cignal™Reporter Assay Kit, SABiosciences Corporation, USA). One day after the transfection the medium can be replaced with 100 μl fresh medium containing 2% FBS, 2 mM L-Glutamine, 100 U/ml Penicillin (PAA), 100 μg/ml Streptomycin (PAA) and anti-TMEFF2 monoclonal antibody in a required concentration. After 6 hours of antibody incubation at 37° C., cells are stimulated with recombinant human/mouse/rat Activin A (R&D Systems, Minneapolis, USA). Activin is diluted to an end concentration of 50 μg/m in the same medium as for antibody incubation and added on. After 18 hours of stimulation (Incubator, 37° C., 5% CO₂) cells are lysed and luciferase activity is determined by using a Dual-Luciferase®Reporter Assay System (Promega Corporation, WI, USA). Luminescence is measured in a 10-second measurement period in a GloMax™ 96 Microplate Luminometer (Promega Corporation) and obtained signals are analyzed according to the assay description.

The data show an increase in the relative luminescence signal following stimulation with Activin, reflecting an Activin induced Smad-regulated signaling pathway activity in the TMEFF2 overexpressing cells (FIG. 10, white bar). The Activin-induced signal is further increased when an anti-TMEFF2 antibody is simultaneously incubated with the antibody PQ01 being most potent.

1. An antibody or antigen-binding fragment thereof specifically binding transmembrane protein with EGF-like and two follistatin-like domains 2 (TMEFF2), which antibody is capable of
(a) increasing Activin induced Smad-regulated signaling pathway activity in TMEFF2 over-expressing CHO cells;
(b) displaying anxiolytic properties in the novelty-induced hypophagia (NIH) paradigm test; and/or
(c) displaying antidepressive properties in the forced swim test (FST).

2. The antibody of claim 1, which is capable of binding an epitope comprising the amino acid sequence EDGHYAR (SEQ ID NO: 13).

3. The antibody of claim 1, wherein the antibody is capable of binding a peptide consisting of the amino acid sequence NTTTTTKSEDGHYAR (SEQ ID NO: 14), a peptide consisting of the amino acid sequence TTKSEDGHYARTDYA (SEQ ID NO: 15) and/or a peptide consisting of EDGHYARTDYAENAN (SEQ ID NO: 16).

4. The antibody of claim 1 comprising in its variable region
(a) at least one complementarity determining region (CDR) of the V_{H} and/or V_{L} variable region amino acid sequences depicted in
(i) FIG. 1 (VH) (SEQ ID NOs: 3, 4, 5); and
(ii) FIG. 1 (VL) (SEQ ID NOs: 6, 7, 6);
(b) an amino acid sequence of the V_{H} and/or V_{L} region as depicted in FIG. 1;
(c) at least one CDR consisting of an amino acid sequence resulted from a partial alteration of any one of the amino acid sequences of (a);
(d) a heavy chain and/or light variable region comprising an amino acid sequence resulted from a partial alteration of the amino acid sequence of (b); or
(e) at least one CDR comprising an amino acid sequence with at least 90% identity to any one of the amino acid sequences of (a).

5. The antibody of claim 1 comprising:
(a) an H-CDR1, H-CDR2 and H-CDR3 with SEQ ID NOs: 3, 4 and 5, respectively; and
(b) an L-CDR1, L-CDR2 and L-CDR3 with SEQ ID NOs: 6, 7 and 8, respectively, or comprising
(c) variants of the H-CDRs 1 to 3 mentioned in (a) in which the sequences of the H-CDRs 1 to 3 taken as a whole are at least 90% homologous to the sequences shown in SEQ ID NOs: 3, 4 and 5 taken as a whole; and
(d) variants of the L-CDRs 1 to 3 mentioned in (b) in which the sequences of the L-CDRs 1 to 3 taken as a whole are at least 90% homologous to the sequences shown in SEQ ID NOs: 6, 7 and 8 taken as a whole.

6. An antibody or antigen-binding molecule which is capable of competing with the antibody of claim 1 for specific binding to TMEFF2.

7. The antibody of claim 1, which is human, humanized or a synthetic human antibody.

8. The antibody of claim 1, which is an Fab or scFv antibody.

9. The antibody or antigen-binding fragment of claim 1, further comprising a penetration enhancing peptide.

10. A polynucleotide encoding at least the variable region of one immunoglobulin chain of the antibody or antigen-binding fragment of claim 1.

11. A vector comprising the polynucleotide of claim 10, which encodes the variable region of the other immunoglobulin chain of said antibody.

12. A host cell comprising the polynucleotide of claim 10.

13. The antibody or antigen-binding fragment of claim 1, which is detectably labeled or attached to a drug.

14. A composition comprising the antibody or antigen-binding fragment of claim 1, wherein the composition is
i) a pharmaceutical composition and further comprises a pharmaceutically acceptable carrier, or
ii) a diagnostic composition, and further comprises reagents conventionally used immuno- or nucleic acid based diagnostic methods.

15. A compound comprising the antibody or antigen-binding fragment of claim 1 for use in treating or preventing an affective and/or anxiety disorder or for diagnosing or screening a subject for the presence or for determining a subject's risk for developing an affective and/or anxiety disorder.

16. The antibody of claim 5, wherein the sequences of the H-CDRs 1 to 3 taken as a whole are at least 95% identical to SEQ ID NOs: 3, 4 and 5 taken as a whole, and the sequences of the L-CDRs 1 to 3 taken as a whole are at least 95% identical to SEQ ID NOs: 6, 7 and B taken as a whole.

17. The antibody or antigen-binding fragment of claim 13, wherein the detectable label is selected from the group consisting of an enzyme, a radioisotope, a fluorophore and a heavy metal.

18. The composition of claim 14, wherein the pharmaceutical composition is formulated for nasal administration or injection.

19. The composition of claim 18, wherein the pharmaceutical composition is formulated for extended release.

20. The compound of claim 15, wherein the affective and/or anxiety disorder is selected from the group consisting of major depression, anxiety, dysthymia, atypical depression, premenstrual dysphoric disorder, seasonal affective disorder, and bipolar disorder.
